(19) United States
(12) Patent Application Publication
(10) Pub. No.: US 2026/0224626 A1
(43) Pub. Date: Aug. 6, 2026

(54) BIOMARKERS RELATED TO IMMUNOTHERAPY EFFICACY

(71) Applicant: Kite Pharma, Inc., Santa Monica, CA (US)

(72) Inventors: Soumya Poddar, Los Angeles, CA (US); Gabriela Balderrama-Gutierrez, Los Angeles, CA (US); Davide Bedognetti, Marina Del Rey, CA (US); Michael D. Mattie, Torrance, CA (US)

(21) Appl. No.: 19/375,481

(22) Filed: Oct. 31, 2025

Related U.S. Application Data
(60) Provisional application No. 63/715,807, filed on Nov. 4, 2024.
(60) Provisional application No. 63/813,797, filed on May 29, 2025.

(51) Int. Cl. A61K 35/17; A61K 40/11; A61K 40/31; A61K 40/42; A61K 45/06; A61P 35/00; C12N 5/00; C12N 5/0783; C12N 15/86; G01N 33/569; G01N 33/68

(52) U.S. Cl. CPC A61K 35/17; A61K 40/11; A61K 40/31; A61K 40/4211; A61K 45/06; A61P 35/00; C12N 5/0018; C12N 5/0087; C12N 5/0636; C12N 15/86; G01N 33/56972; G01N 33/6872; A61K 2239/13; A61K 2239/31; C12N 2501/2302; C12N 2501/2304; C12N 2501/2305; C12N 2501/2313; C12N 2501/51; C12N 2501/515; C12N 2510/00; C12N 2740/10043; C12N 2740/15043; G01N 2333/4703

(57) ABSTRACT

The disclosure relates to methods for predicting a likelihood of a relapse to a cancer in a patient following treatment of the patient with an immunotherapy product, and methods for improving efficacy of an immunotherapy product for a patient with a likelihood of a relapse to a cancer following treatment with an immunotherapy product.

### CROSS-REFERENCE TO RELATED APPLICATIONS

The present application claims the priority benefit of U.S. Provisional Application No. 63/715,807, filed Nov. 4, 2024, and of U.S. Provisional Application No. 63/813,797, filed May 29, 2025, which are hereby incorporated by reference in their entireties.

### BACKGROUND

Human cancers are by their nature comprised of normal cells that have undergone a genetic or epigenetic conversion to become abnormal cancer cells. In doing so, cancer cells begin to express proteins (including, but not limited to, antigens) that are distinct from those expressed by normal cells. These aberrant tumor antigens may be used by the body's innate immune system to specifically target and kill cancer cells. However, cancer cells employ various mechanisms to prevent immune cells, such as T and B lymphocytes, from successfully targeting cancer cells.

Human T cell therapies rely on enriched or modified human T cells to target and kill cancer cells in a patient. To increase the ability of T cells to target and kill a particular cancer cell, methods have been developed to engineer T cells to express constructs which direct T cells to a particular target cancer cell. For example, chimeric antigen receptors (CARs) and T Cell Receptors (TCRs), which comprise binding domains capable of interacting with a particular tumor antigen, allow T cells to target and kill cancer cells that express the particular tumor antigen. There is a need to understand how pre-treatment attributes of human T cell therapies (e.g., expression levels of certain genes) influence treatment outcomes, including safety outcomes of immunotherapy.

### SUMMARY

It is to be understood that the disclosure is not limited in its application to the details set forth in the following embodiments, claims, description and figures. The disclosure is capable of other embodiments and of being practiced or carried out in numerous other ways.

Provided herein are methods that involve assessing particular parameters, e.g., expression of specific biomarkers or analytes, that can be correlated with an outcome, such as a therapeutic outcome, including a response, such as a complete response (CR) or a partial response (PR); or a safety outcome (e.g. an adverse event), such as a development of a toxicity, for example, neurotoxicity or CRS, after administration of immunotherapy (e.g., cell therapy). Also provided are methods to assess the likelihood of response and/or likelihood of risk of toxicity, based on assessment of the parameters, such as expression of biomarkers or analytes in the patient. Also provided are immunotherapies (e.g., T cells, non-T cells, TCR-based therapies, CAR-based therapies, bispecific T-cell engagers (BiTEs), and/or immune checkpoint blockade), including methods and uses of cells (e.g., engineered T cells) and/or compositions thereof, for the treatment of subjects having a disease or condition, which generally is or includes a cancer or a tumor, such as a leukemia or a lymphoma. In some aspects, the methods and uses provide for or achieve improved response and/or more durable responses or efficacy and/or a reduced risk of toxicity or other side effects, in subjects treated with some methods, as compared to certain alternative methods. In some embodiments, the methods comprise the administration of specified numbers or relative numbers of the engineered cells, the administration of defined ratios of particular types of the cells, treatment of particular patient populations, such as those having a particular risk profile, staging, and/or prior treatment history, administration of additional therapeutic agents and/or combinations thereof.

An embodiment of the disclosure relates to a method for manufacturing transformed lymphocytes, including: measuring an expression level of GATA3 in a sample of lymphocytes collected from a subject; reducing an activity of or an amount of GATA3+ CD8+ cells from the sample of lymphocytes if the expression level of GATA3 in the sample of lymphocytes is above a control value; and incubating the sample of lymphocytes with a polynucleotide vector to transform the sample of lymphocytes to produce transformed lymphocytes.

An embodiment of the disclosure relates to a method of predicting a likelihood of a relapse to a cancer in a patient treated with a cell therapy product including: measuring a pre-treatment level of GATA3+CD8+ cells in a sample of lymphocytes collected from the subject pre-treatment; determining the likelihood of relapse in the patient from the pre-treatment level of GATA3+CD8+ cells, where an elevated pre-treatment level of GATA3+CD8+ cells as compared to a pre-treatment control value from patients with the same cancer and same prior lines of therapy, if any, is indicative of an increased likelihood of relapse in the patient, and where the patient is treated with the cell therapy product after the measuring the pre-treatment level of GATA3+CD8+ cells.

An embodiment of the disclosure relates to a method of predicting a likelihood of a relapse to a cancer in a patient treated with a cell therapy product including: measuring a pre-treatment Th1-Th2 index in a sample of lymphocytes collected from the subject pre-treatment; determining the likelihood of relapse in the patient from the pre-treatment Th1-Th2 index, where a pre-treatment Th1-Th2 index below 0.0 is indicative of an increased likelihood of relapse in the patient, and where the patient is treated with the cell therapy product after the measuring the pre-treatment Th1-Th2 index.

An embodiment of the disclosure relates to a method for treating a malignancy in a patient including: measuring an expression level of GATA3 in a cell therapy product; determining whether the patient should be administered an effective dose of the cell therapy product, or an effective dose of the cell therapy product and an inhibitor of a Th2 cytokine based on the measuring the expression level of GATA3; and administering the effective dose of the cell therapy product, or the effective dose of the cell therapy product and the inhibitor of a Th2 cytokine based on the determining step, where the patient is administered the effective dose of the cell therapy product if the expression level of GATA3 is at or below a control value, and where the patient is administered the effective dose of the cell therapy product and the inhibitor of a Th2 cytokine if the expression level of GATA3 is above the control value.

In one embodiment, the immunotherapy is T cell therapy. In some embodiments, the T cell therapy includes an adoptive cell therapy. In certain embodiments, the adoptive cell therapy is selected from tumor-infiltrating lymphocyte (TIL) immunotherapy, autologous cell therapy, engineered autologous cell therapy (eACT), and allogeneic T cell transplantation. In one particular embodiment, the eACT includes administration of engineered antigen specific chimeric antigen receptor (CAR) positive (+) T cells. In another embodiment, the eACT includes administration of engineered antigen specific T cell receptor (TCR) positive (+) T cells. In one embodiment, the immunotherapy is CAR T cell or TCR T cell therapy. In one embodiment, the immunotherapy is anti-CD19 CAR T cell therapy.

The following are non-limiting embodiments of the disclosure.

An embodiment of the disclosure relates to a method for manufacturing transformed lymphocytes, including: measuring an expression level of GATA3 in a sample of lymphocytes collected from a subject; reducing an activity of or an amount of GATA3+ CD8+ cells from the sample of lymphocytes if the expression level of GATA3 in the sample of lymphocytes is above a control value; and incubating the sample of lymphocytes with a polynucleotide vector to transform the sample of lymphocytes to produce transformed lymphocytes.

An embodiment of the disclosure relates to the method above, where reducing the activity or the amount of GATA3+ CD8+ cells from the sample of lymphocytes includes depleting cells expressing CD45RO from the sample of lymphocytes.

An embodiment of the disclosure relates to the method above, where depleting the cells expressing CD45RO from the sample of lymphocytes includes: contacting the sample of lymphocytes with an anti-hCD45RO antibody and bead conjugate so as to generate a bead-bound population of cells expressing CD45RO; and separating the bead-bound population of cells expressing CD45RO from the sample of lymphocytes.

An embodiment of the disclosure relates to the method above, where reducing the activity or the amount of GATA3+ CD8+ cells from the sample of lymphocytes includes incubating the sample of lymphocytes with a Th2 cytokine inhibitor.

An embodiment of the disclosure relates to the method above, where the Th2 cytokine inhibitor is selected from the group consisting of an anti-IL4 antibody, an anti-IL5 antibody, and an anti-IL13 antibody. More specifically, the anti-IL4 antibody is dupilumab (Dupixent), the anti-IL5 antibody is mepolizumab, relizumab, or benralizumab, and an anti-IL13 antibody is trakolinumab.

An embodiment of the disclosure relates to the method above, where the Th2 cytokine inhibitor is an anti-IL4 antibody.

An embodiment of the disclosure relates to the method above, further including activating the sample of lymphocytes before incubating the sample of lymphocytes with the polynucleotide vector by stimulating the sample of lymphocytes with at least one T cell stimulating agent.

An embodiment of the disclosure relates to the method above, where the at least one T cell stimulating agent includes an anti-CD3 antibody, an anti-CD28 antibody, or Interleukin-2, and combinations thereof.

An embodiment of the disclosure relates to the method above, where the activating is carried out in a closed system.

An embodiment of the disclosure relates to the method above, further including: culturing the sample including the transformed lymphocytes for up to 72 hours before the lymphocytes are harvested to produce a harvested sample.

An embodiment of the disclosure relates to the method above, where the transformed lymphocytes are cultured for less than 48 hours before being harvested.

An embodiment of the disclosure relates to the method above, where the transformed lymphocytes are cultured for less than 36 hours before being harvested.

An embodiment of the disclosure relates to the method above, further including, administering the transformed lymphocytes to a subject in need thereof or freezing the transformed lymphocytes.

An embodiment of the disclosure relates to the method above, where the polynucleotide vector is a viral vector.

An embodiment of the disclosure relates to the method above, where the viral vector is a retroviral vector or a lentiviral vector.

An embodiment of the disclosure relates to the method above, where the polynucleotide vector encodes a chimeric antigen receptor (CAR) or a T cell receptor (TCR).

An embodiment of the disclosure relates to the method above, where the CAR or the TCR recognizes a tumor antigen.

An embodiment of the disclosure relates to the method above, where the tumor antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, Fc receptor-like protein 5, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, TACI, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), EphA2, GPC2, CD133, LGR5, TROP2, CD146, CLDN3, CLDN4, CLDN6, CLDN9, CLDN18.2, CD70, TnMUC1, Alkaline phosphatase (placental type), MUC16, MUC17, MART1, Pmel17, Melanoma-associated chondroitin sulfate proteoglycan, as well as any derivate or variant of these antigens.

An embodiment of the disclosure relates to a population of cells prepared by the methods above.

An embodiment of the disclosure relates to a pharmaceutical composition including the population of cells disclosed of above.

An embodiment of the disclosure relates to a method for administering T cells to a subject, including injecting to the subject a harvested sample prepared by the methods above or the pharmaceutical composition disclosed above.

An embodiment of the disclosure relates to the method above, where the subject has cancer.

An embodiment of the disclosure relates to the method above, where the cancer is a lung cancer, a GI cancer, a breast cancer, a gynecologic malignancy, a genitourinary malignancy, a neurologic tumor, a melanoma, a sarcoma, a pediatric cancer, an endocrine malignancy, Kaposi sarcoma, a Non-Hodgkin's Lymphoma, or mesothelioma.

An embodiment of the disclosure relates to the method above, where the cancer is a B cell malignancy.

An embodiment of the disclosure relates to the method above, where the cancer is Non-Hodgkin's Lymphomas (NHL), Diffuse Large B Cell Lymphoma (DLBCL), Small lymphocytic lymphoma (SLL/CLL), Mantle cell lymphoma (MCL), Follicular lymphoma (FL), Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, Lymphoblastic lymphoma, acute myeloid leukemia, or multiple myeloma.

An embodiment of the disclosure relates to a method of predicting a likelihood of a relapse to a cancer in a patient treated with a cell therapy product including: measuring a pre-treatment level of GATA3+CD8+ cells in a sample of lymphocytes collected from the subject pre-treatment; determining the likelihood of relapse in the patient from the pre-treatment level of GATA3+CD8+ cells, where an elevated pre-treatment level of GATA3+CD8+ cells as compared to a pre-treatment control value from patients with the same cancer and same prior lines of therapy, if any, is indicative of an increased likelihood of relapse in the patient, and where the patient is treated with the cell therapy product after the measuring the pre-treatment level of GATA3+CD8+ cells. In such an embodiment, the term “pre-treatment” refers to before the patient is treated with the cell therapy product.

An embodiment of the disclosure relates to the method above, where the cancer is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.

An embodiment of the disclosure relates to the method above, where the cell therapy product is CAR T or TCR T cell therapy that recognizes a target antigen.

An embodiment of the disclosure relates to the method above, where the cell therapy product is autologous or allogeneic.

An embodiment of the disclosure relates to the method above, where the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), as well as any derivate or variant of these antigens.

An embodiment of the disclosure relates to a method of predicting a likelihood of a relapse to a cancer in a patient treated with a cell therapy product including: measuring a pre-treatment Th1-Th2 index in a sample of lymphocytes collected from the subject pre-treatment; determining the likelihood of relapse in the patient from the pre-treatment Th1-Th2 index, where a pre-treatment Th1-Th2 index below 0.0 is indicative of an increased likelihood of relapse in the patient, and where the patient is treated with the cell therapy product after the measuring the pre-treatment Th1-Th2 index. In such an embodiment, the term “pre-treatment” refers to before the patient is treated with the cell therapy product.

An embodiment of the disclosure relates to the method above, where the cancer is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.

An embodiment of the disclosure relates to the method above, where the cell therapy product is CAR T or TCR T cell therapy that recognizes a target antigen.

An embodiment of the disclosure relates to the method above, where the cell therapy product is autologous or allogeneic.

An embodiment of the disclosure relates to the method above, where the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), as well as any derivate or variant of these antigens.

An embodiment of the disclosure relates to a method for treating a malignancy in a patient including: measuring an expression level of GATA3 in a cell therapy product; determining whether the patient should be administered an effective dose of the cell therapy product, or an effective dose of the cell therapy product and an inhibitor of a Th2 cytokine based on the measuring the expression level of GATA3; and administering the effective dose of the cell therapy product, or the effective dose of the cell therapy product and the inhibitor of a Th2 cytokine based on the determining step, where the patient is administered the effective dose of the cell therapy product if the expression level of GATA3 is at or below a control value, and where the patient is administered the effective dose of the cell therapy product and the inhibitor of a Th2 cytokine if the expression level of GATA3 is above the control value.

An embodiment of the disclosure relates to the method above, where the inhibitor of a Th2 cytokine is selected from the list consisting of an IL-4 antagonist, an IL-5 antagonist, and an IL-13 antagonist.

An embodiment of the disclosure relates to the method above, where the inhibitor of a Th2 cytokine is an anti-IL-4 antibody, an anti-IL-5 antibody, or an anti-IL-13 antibody. More specifically, the anti-IL4 antibody is dupilumab (Dupixent), the anti-IL5 antibody is mepolizumab, relizumab, or benralizumab, and an anti-IL13 antibody is trakolinumab.

An embodiment of the disclosure relates to the method above, where the cell therapy product is CAR T or TCR T cell therapy that recognizes a target antigen.

An embodiment of the disclosure relates to the method above, where the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), as well as any derivate or variant of these antigens.

An embodiment of the disclosure relates to the method above, where the cell therapy product expresses a chimeric antigen receptor including a CD28 co-stimulatory domain.

An embodiment of the disclosure relates to the method above, where the patient has been diagnosed with a cancer/tumor selected from the group consisting of a solid tumor, sarcoma, carcinoma, lymphoma, multiple myeloma, Hodgkin's Disease, non-Hodgkin's lymphoma (NHL), primary mediastinal large B cell lymphoma (PMBCL), diffuse large B cell lymphoma (DLBCL) (not otherwise specified), follicular lymphoma (FL), DLBCL arising from FL, transformed follicular lymphoma, high grade B cell lymphoma, splenic marginal zone lymphoma (SMZL), chronic or acute leukemia, acute myeloid leukemia, chronic myeloid leukemia, acute lymphoblastic leukemia (ALL) (including non T cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma), monoclonal gammapathy of undetermined significance (MGUS), plasmacytomas (e.g., plasma cell dyscrasia, solitary myeloma, solitary plasmacytoma, extramedullary plasmacytoma, and multiple plasmacytoma), systemic amyloid light chain amyloidosis, POEMS syndrome (also known as Crow-Fukase syndrome, Takatsuki disease, and PEP syndrome), head and neck cancers, cervical cancers, ovarian cancers, non-small cell lung carcinomas, hepatocellular carcinomas, prostate cancers, breast cancers, or a combination thereof.

An embodiment of the disclosure relates to the method above, where the malignancy is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.

An embodiment of the disclosure relates to the method above, where the cell therapy product is selected from axicabtagene ciloleucel, brexucabtagene autoleucel, tisagenlecleucel, lisocabtagene maraleucel, and bb2121.

### BRIEF DESCRIPTION OF THE DRAWINGS

FIGS. 1A, 1B, and 1C are schematics and graphs showing that a subset of product samples analyzed represents the clinical response in ZUMA1, according to an embodiment of the disclosure.

FIGS. 2A, 2B, 2C, 2D, and 2E are a schematic and graphs showing that in vitro measurement of Th1 and Th2 cytokines correlates with clinical response, according to an embodiment of the disclosure.

FIGS. 3A, 3B, 3C, 3D, and 3E are schematics and graphs showing the scRNAseq of products identify dysfunctional immune regulation associated with disease progression, according to an embodiment of the disclosure.

FIGS. 4A, 4B, 4C, 4D, 4E, and 4F are schematics and graphs showing that corresponding clusters of scATACseq validate findings on GATA3+CD8+ dysfunctional T-cells, according to an embodiment of the disclosure.

FIGS. 5A, 5B, 5C, and 5D are graphs showing quantification of dysfunctional GATA3+CD8+ T cells through immunophenotyping, according to an embodiment of the disclosure.

FIGS. 6A, 6B, 6C, 6D, and 6E are schematics and graphs showing that Axi-cel products administered to ZUMA1 patients showed similar efficacy in a systemic human B-cell lymphoma in vivo model, according to an embodiment of the disclosure.

FIGS. 7A, 7B, 7C, and 7D are graphs showing that the Th1-Th2 index measured in vivo associate with clinical response, according to an embodiment of the disclosure.

FIG. 8 is a table showing final panel iteration with fluorophore assignment and marker specificity for detecting T cell subtypes, according to an embodiment of the disclosure.

### DETAILED DESCRIPTION

The present disclosure is related to methods for predicting a likelihood of a relapse to a cancer in a patient, methods for predicting a likelihood of a durable response to a cell therapy product in a patient in need thereof, and methods for treating a malignancy in a patient.

### Definitions

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed disclosure.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Systeme International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. Exemplary routes of administration for the compositions disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering may also be performed, for example, once, a plurality of times, and/or over one or more extended periods. In one embodiment, the CAR T cell treatment is administered via an “infusion product” comprising CAR T cells.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (Clq) of the classical complement system.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An “antigen binding molecule,” “antigen binding portion,” or “antibody fragment” refers to any molecule that comprises the antigen binding parts (e.g., CDRs) of the antibody from which the molecule is derived. An antigen binding molecule may include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In some embodiments, the antigen binding molecule binds to CD19. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocks a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

In one embodiment, the CAR T cell treatment comprises “axicabtagene ciloleucel treatment”. “Axicabtagene ciloleucel treatment” consists of a single infusion of anti-CD19 CAR transduced autologous T cells administered intravenously at a target dose of 2×10⁶ anti-CD19 CAR T cells/kg. For subjects weighing greater than 100 kg, a maximum flat dose of 2×10⁸ anti-CD19 CAR T cells may be administered. The anti-CD19 CAR T cells are autologous human T cells that have been engineered to express an extracellular single-chain variable fragment (scFv) with specificity for CD19 linked to an intracellular signaling part comprised of signaling domains from CD28 and CD3ζ ((CD3-zeta) molecules arranged in tandem anti-CD19 CAR vector construct has been designed, optimized and initially tested at the Surgery Branch of the National Cancer Institute (NCI, IND 13871) (Kochenderfer et al, J Immunother. 2009; 32(7):689-702; Kochenderfer et al, Blood. 2010; 116(19):3875-86). The scFv is derived from the variable region of the anti-CD19 monoclonal antibody FMC63 (Nicholson et al, Molecular Immunology. 1997; 34(16-17):1157-65). A portion of the CD28 costimulatory molecule is added, as murine models suggest this is important for the anti-tumor effect and persistence of anti-CD19 CAR T cells (Kowolik et al, Cancer Res. 2006; 66(22):10995-1004). The signaling domain of the CD3-zeta chain is used for T cell activation. These fragments were cloned into the murine stem cell virus-based (MSGV1) vector, utilized to genetically engineer the autologous T cells. The CAR construct is inserted into the T cells' genome by retroviral vector transduction. Briefly, peripheral blood mononuclear cells (PBMCs) are obtained by leukapheresis and Ficoll separation. Peripheral blood mononuclear cells are activated by culturing with an anti-CD3 antibody in the presence of recombinant interleukin 2 (IL-2). Stimulated cells are transduced with a retroviral vector containing an anti-CD19 CAR gene and propagated in culture to generate sufficient engineered T cells for administration. In some embodiments, the CAR T cell therapy is Yescarta® (axicabtagene ciloleucel). In some embodiments, the CAR T cell therapy is Tecartus® (brexucabtagene autoleucel).

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer is synonymous with malignancy. Examples of cancers that may be treated by the methods disclosed herein include, but are not limited to, cancers of the immune system including lymphoma, leukemia, myeloma, and other leukocyte malignancies. In some embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In some embodiments, the cancer is multiple myeloma. In some embodiments, the cancer is NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

An “anti-tumor effect” as used herein, refers to a biological effect that may present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect may also refer to the prevention of the occurrence of a tumor, e.g., a vaccine.

A “cytokine,” as used herein, refers to a non-antibody protein that is released by one cell in response to contact with a specific antigen, where the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perform. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

As used herein, “chimeric receptor” refers to an engineered surface expressed molecule capable of recognizing a particular molecule. Chimeric antigen receptors (CARs) and engineered T cell receptors (TCRs), which comprise binding domains capable of interacting with a particular tumor antigen, allow T cells to target and kill cancer cells that express the particular tumor antigen. In one embodiment, the T cell treatment is based on T cells engineered to express a chimeric antigen receptor (CAR) or a T cell receptor (TCR), which comprises (i) an antigen binding molecule, (ii) a costimulatory domain, and (iii) an activating domain. The costimulatory domain may comprise an extracellular domain, a transmembrane domain, and an intracellular domain, where the extracellular domain comprises a hinge domain, which may be truncated.

A “therapeutically effective amount,” “effective dose,” “effective amount,” or “therapeutically effective dosage” of a therapeutic agent, e.g., engineered CAR T cells, small molecules, “agents” described in the specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms may be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens can be determined empirically by testing in known in vitro or in vivo (e.g., animal model) systems.

The term “combination” refers to either a fixed combination in one dosage unit form, or a combined administration where a compound of the present disclosure and a combination partner (e.g. another drug as explained below, also referred to as “therapeutic agent” or “agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment.

In certain embodiments, a reference or control is a patient population which has the same type of cancer, has some of the same demographic attributes, has received the same line of treatment, has received the same prior lines of cancer treatment and/or has other similarities as an individual patient under treatment, treatment monitoring and/or other consideration.

The terms “control value” and “reference value” refer to a historical median value of the characteristic under assessment as observed in subjects from the same cancer and same prior lines of therapy, if any, as the patient undergoing the assessment. For example, if the expression level of GATA3 in a sample of lymphocytes collected from the patient is being assessed, that expression level of GATA3 will be compared to the historical median value of the expression level of GATA3 in samples of lymphocytes collected from other subjects having the same cancer and same prior lines of therapy, if any, as the patient.

The term “Th1 cytokine” refers to a cytokine produced by a T helper 1 (Th1) cell. Th1 cells stimulate cellular immune response, participate in the inhibition of macrophage activation and stimulate B cells to produce IgM, IgG1. Non-limiting examples of cytokines produced by Th1 cells include IFN-γ and IL-2.

The term “Th2 cytokine” refers to a cytokine produced by a T helper 2 (Th2) cell. Th2 cells stimulate humoral immune response, promote B cell proliferation and induce antibody production. Non-limiting examples of cytokines produced by Th2 cells include IL-4, IL-5, and IL-13.

The term “Th1-Th2 index” refers to a measurement of the levels of Th1 cytokines versus the levels of Th2 cytokines in a stimulated CAR-T cell product that is to be administered to a patient in need. The Th1-Th2 index informs on a correlation between Th1 and Th2 cytokines and clinical response of the patient to the CAR-T cell product. The Th1-Th2 index is calculated as follows:

Th1-Th2 index=(IFNg+IL-2)/2−(IL-4+IL-5+IL-13)/3

In the above formula, the measured levels of each of IFNg, IL-2, IL-4, IL-5, and IL-13 are normalized between 0 and 1 for equal weightage prior to application of the formula. A Th1-Th2 index score below 0 is correlated with an increased likelihood of relapse following treatment of the patient with the CAR-T cell product.

The term “pre-treatment” refers to a time frame prior to, but relatively contemporaneous with, the administration of a line of cancer therapy which may be the first line of treatment for a cancer in a patient, a second line of treatment for cancer in a patient, a third line of treatment for cancer in a patient or a subsequent line of treatment for cancer in a patient. The term “pre-treatment” also refers to a time frame before a patient is treated with a cell therapy product.

The term “durable response” refers to a patient having a progression-free survival (PFS) exceeding three times the median PFS of the whole population of patients treated with the same drug(s) in the same trial.

The terms “product” or “infusion product” are used interchangeably herein and refer to the T cell composition that is administered to the subject in need thereof. Typically, in CAR T-cell therapy, the T cell composition is administered as an infusion product.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory TSCM cells, like naive cells, are CD45RO−, CCR7+, CD45RA+, CD62L+ (L-selectin), CD27+, CD28+ and IL-7Rα+, but they also express large amounts of CD95, IL-2Rβ, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T cells (Tregs, suppressor T cells, or CD4+CD25+ regulatory T cells), Natural Killer T cells (NKT) and Gamma Delta T cells. B-cells, on the other hand, play a principal role in humoral immunity (with antibody involvement).

Furthermore, each type of T cells can be characterized with cell surface markers, as well known in the art. For instance, naïve T cells can be characterized as CCR7+, CD45RO−, and CD95−. Additional markers for naïve T cell include CD45RA+, CD62L+, CD27+, CD28+, CD127+, CD132+, CD25−, CD44−, and HLA-DR−. Surface markers to stem memory T cells (Tscm) include, without limitation, CD45RO−, CCR7+, CD45RA+, CD62L+ (L-selectin), CD27+, CD28+, IL-7Ra+, CD95+, IL-2RP+, CXCR3+, and LFA−. Surface markers for effector memory T cells (Tem) include, without limitation, CCR7−, CD45RO+ and CD95+. Additional marker for effector memory T cells is IL-2Rβ+. For central memory T cells (Tcm), suitable markers include CD45RO+, CD95+, IL-2Rβ+, CCR7+ and CD62L+. For effector T cells (Teff), suitable markers include CD45RA+, CD95+, IL-2Rβ+, CCR7− and CD62L−, without limitation.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing, or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. Nos. 7,741,465, 6,319,494, 5,728,388, International Publication No. WO 2008/081035, International Publication No. WO 2015/20096, International Publication No. WO 2016/191756, International Publication No. WO 2016/191755, International Publication No. WO 2019/079564, and International Publication No. WO 2021/092290, each of which are herein incorporated in their entireties. In some embodiments, the immunotherapy comprises CAR T cell treatment. In some embodiments, the CAR T cell treatment product is administered via infusion.

The T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy, as well as methods for making CAR T cells for cell therapy are disclosed in U.S. Patent Publication No. 2013/0287748, International Publication No. WO 2015/120096, International Publication No. WO 2016/191756, International Publication No. WO 2016/191755, International Publication No. WO 2019/079564, International Publication No. WO 2021/092290, and International Publication WO 2023/230276, each of which are herein incorporated by reference in their entirety.

The term “engineered Autologous Cell Therapy,” or “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR). CAR positive (+) T cells are engineered to express an extracellular single chain variable fragment (scFv) with specificity for a particular tumor antigen linked to an intracellular signaling part comprising at least one costimulatory domain and at least one activating domain. The CAR scFv may be designed to target, for example, CD19, which is a transmembrane protein expressed by cells in the B cell lineage, including all normal B cells and B cell malignances, including but not limited to diffuse large B-cell lymphoma (DLBCL) not otherwise specified, primary mediastinal large B-cell lymphoma, high grade B-cell lymphoma, and DLBCL arising from follicular lymphoma, NHL, CLL, and non-T cell ALL. Example CAR T cell therapies and constructs are described in U.S. Patent Publication Nos. 2013/0287748, 2014/0227237, 2014/0099309, and 2014/0050708, and these references are incorporated by reference in their entirety.

A “patient” or a “subject” as used herein includes any human who is afflicted with a cancer (e.g., a lymphoma or a leukemia). The terms “subject” and “patient” are used interchangeably herein.

As used herein, the term “in vitro cell” refers to any cell which is cultured ex vivo. In particular, an in vitro cell may include a T cell. The term “in vivo” means within the patient.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex that specifically binds with a cognate stimulatory ligand present on an antigen present cell. A “stimulatory ligand” is a ligand that when present on an antigen presenting cell (e.g., an APC, a dendritic cell, a B-cell, and the like) may specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. In certain embodiments, a co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD33, CD45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD11a, CD11b, CD11c, CD11d, CDS, CEACAM1, CRT AM, DAP-10, DNAM1 (CD226), Fc gamma receptor, GADS, GITR, HVEM (LIGHTR), IA4, ICAM-1, ICOS, Ig alpha (CD79a), IL2R beta, IL2R gamma, IL7R alpha, integrin, ITGA4, ITGA6, ITGAD, ITGAE, ITGAL, ITGAM, ITGAX, ITGB2, ITGB7, ITGB1, KIRDS2, LAT, LFA-1, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission. In some embodiments, the treatment may be prophylactic, in which case the treatment is administered before any symptoms of the condition are observed. The term “prophylaxis” as used herein means the prevention of or protective treatment for a disease or disease state. Prevention of a symptom, disease, or disease state may include reduction (e.g., mitigation) of one or more symptoms of the disease or disease state, e.g., relative to a reference level (e.g., the symptom(s) in a similar subject not administered the treatment). Prevention may also include delaying onset of one or more symptoms of the disease or disease state, e.g., relative to a reference level (e.g., the onset of the symptom(s) in a similar subject not administered the treatment). In embodiments, a disease is a disease described herein. In some embodiments, the disease is cancer. In some embodiments, the diseased state is CRS or neurotoxicity. In some embodiments, indicators of improvement or successful treatment include determination of the failure to manifest a relevant score on toxicity grading scale (e.g. CRS or neurotoxicity grading scale), such as a score of less than 3, or a change in grading or severity on the grading scale as discussed herein, such as a change from a score of 4 to a score of 3, or a change from a score of 4 to a score of 2, 1 or 0.

As used herein, “myeloid cells” are a subgroup of leukocytes that includes granulocytes, monocytes, macrophages, and dendritic cells.

In one embodiment, the terms “high” and “low” mean “above” and “below” the median value for a representative population of subjects. In one embodiment, the terms mean in the upper or lower quartiles, respectively. Both the mean and the quartile distribution may be determined by one of ordinary skill in the art by routine methods.

As used herein, the term “quartile” is a statistical term describing a division of observations into four defined intervals based upon the values of the data and how they compare to the entire set of observations.

As used herein, the term “Study day 0” is defined as the day the subject received the first CAR T cell infusion. The day prior to study day 0 will be study day −1. Any days after enrollment and prior to study day −1 will be sequential and negative integer-valued.

As used herein, the term “durable response” refers to the subjects who were in ongoing response at least by one year follow up post CAR T cell infusion. In one embodiment, “duration of response” is defined as the time from the first objective response to disease progression or to death due to disease relapse.

As used herein, the term “relapse” refers to the subjects who achieved a complete response (CR) or partial response (PR) and subsequently experienced disease progression.

As used herein, the term “non-response” refers to the subjects who had never experienced CR or PR post CAR T cell infusion, including subjects that with stable disease (SD) and progressive disease (PD).

As used herein, the term “objective response” refers to complete response (CR), partial response (PR), or non-response. It may be assessed per revised IWG Response Criteria for Malignant Lymphoma (Cheson et al., J Clin Oncol. 2007; 25(5):579-86)

As used herein, the term “complete response” refers to complete resolution of disease, which becomes not detectable by radio-imaging and clinical laboratory evaluation. No evidence of cancer at a given time.

As used herein, the term “partial response” refers to a reduction of greater than 30% of tumor without complete resolution.

As used herein “objective response rate” (ORR) is determine per International Working Group (IWG) 2007 criteria (Cheson et al. J Clin Oncol. 2007; 25(5):579-86).

As used herein “progression-free survival (PFS)” may be defined as the time from the T cell infusion date to the date of disease progression or death from any cause. Progression is defined per investigator's assessment of response as defined by IWG criteria (Cheson et al., J Clin Oncol. 2007; 25(5):579-86).

The term “overall survival (OS)” may be defined as the time from the T cell infusion date to the date of death from any cause.

As used herein, the expansion and persistence of CAR T cells in peripheral blood may be monitored by qPCR analysis, for example using CAR-specific primers for the scFv portion of the CAR (e.g., heavy chain of a CD19 binding domain) and its hinge/CD28 transmembrane domain. Alternatively, it may be measured by enumerating CAR cells/unit of blood volume.

As used herein, the scheduled blood draw for CAR T cells may be before CAR T cell infusion, Day 7, Week 2 (Day 14), Week 4 (Day 28), Month 3 (Day 90), Month 6 (Day 180), Month 12 (Day 360), and Month 24 (Day 720).

As used herein, the “peak of CAR T cell” is defined as the maximum absolute number of CAR+ PBMC/μL in serum attained after Day 0.

As used herein, the “time to Peak of CAR T cell” is defined as the number of days from Day 0 to the day when the peak of CAR T cell is attained.

As used herein, the “Area Under Curve (AUC) of level of CAR T cell from Day 0 to Day 28” is defined as the area under the curve in a plot of levels of CAR T cells against scheduled visits from Day 0 to Day 28. This AUC measures the total levels of CAR T cells overtime.

As used herein, the scheduled blood draw for cytokines is before or on the day of conditioning chemotherapy (Day −5), Day 0, Day 1, Day 3, Day 5, Day 7, every other day if any through hospitalization, Week 2 (Day 14), and Week 4 (Day 28).

As used herein, the “baseline” of cytokines is defined as the last value measured prior to conditioning chemotherapy.

As used herein, the fold change from baseline at Day X is defined as

$$\frac{Cytokine level at Day X - Baseline}{Baseline}$$

As used herein, the “peak of cytokine post baseline” is defined as the maximum level of cytokine in serum attained after baseline (Day −5) up to Day 28.

As used herein, the “time to peak of cytokine” post CAR T cell infusion is defined as the number of days from Day 0 to the day when the peak of cytokine was attained.

As used herein, the “Area Under Curve (AUC) of cytokine levels” from Day −5 to Day 28 is defined as the area under the curve in a plot of levels of cytokine against scheduled visits from Day −5 to Day 28. This AUC measures the total levels of cytokine overtime. Given the cytokine and CAR+ T cell are measured at certain discrete time points, the trapezoidal rule may be used to estimate the AUCs.

As used herein, treatment-emergent adverse events (TEAEs) are defined as adverse events (AE) with onset on or after the first dose of conditioning chemotherapy. Adverse events may be coded with the Medical Dictionary for Regulatory Activities (MedDRA) version 22.0 and graded using the National Cancer Institute (NCI) Common Terminology Criteria for Adverse Events (CTCAE) version 4.03. Cytokine Release Syndrome (CRS) events may be graded on the syndrome level per Lee and colleagues (Lee et al, 2014 Blood. 2014; 124(2):188-95. Individual CRS symptoms may be graded per CTCAE 4.03. Neurologic events may be identified with a search strategy based on known neurologic toxicities associated with CAR T immunotherapy, as described in, for example, Topp, M S et al. Lancet Oncology. 2015; 16(1):57-66.

Various aspects of the disclosure are described in further detail in the following subsections.

### Characterization of the Serum Protein Profile of Immunotherapy Cancer Patients

In some embodiments, the present disclosure provides methods to characterize the serum proteomic profile of a cancer patient prior to treatment with immunotherapy and/or pre-conditioning. In one embodiment, immunotherapy is selected from treatment with a chimeric receptor therapy (e.g., YESCARTA™ axicabtagene ciloleucel (axi-cel), TECARTUS™-brexucabtagene autoleucel/KTE-X19, KYMRIAH™ (tisagenlecleucel), etc), TCR, TIL, immune check point inhibitors, among others. In one embodiment, the immunotherapy product comprises autologous or allogeneic CAR T cells. In one embodiment, the immunotherapy comprises T-Cell Receptor-modified T cells. In one embodiment, the immunotherapy comprises tumor infiltrating lymphocytes (TILs). In one embodiment, the immunotherapy product comprises Induced Pluripotent Stem Cells (iPSCs). As described herein, in some embodiments, the serum protein characteristics are obtained through pre-specified protein sets and analyzed through OPI and machine learning models. In some embodiments, the serum levels may be measured by ELISA. In some embodiments, the serum protein profiles associate with adverse events of chimeric receptor therapy (e.g., axicabtagene ciloleucel (axi-cel)) and may be used to predict adverse events in response to all immunotherapies (e.g., T cells, non-T cells, TCR-based therapies, CAR-based therapies, bispecific T-cell engagers (BiTEs), and/or immune checkpoint blockade).

In one embodiment, a high level of biomarkers is a level at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, or at least 100 fold over median or a historical value. In one embodiment, the expression levels of protein biomarker(s) are high or low, respectively, when they fall 0-0.1%, 0.1%-0.5%, 0.5%-1.0%, 1-5%, 5-10%, 10-15%, 15-20%, 20-25%, 25-30%, 30-35%, 35-40%, 40-45%, 45-50%, 50-55%, 55-60%, 60-65%, 65-70%, etc. 95%-100%) above or below, respectively, the median or the values specified above. All listed values may be modified by the term “above.”

In one embodiment, the disclosure provides a method of treating a subject with immunotherapy having a high tumor burden, wherein the immune activation mediated stress in the subject is reduced by administering one or more agents or treatments that result in a reduced inflammation (e.g., lower cytokine induction in the blood) and/or by using an alternative lymphodepleting regimen that does not comprise the administration of 500-600 mg/m²/day of cyclophosphamide and 30 mg/m²/day of fludarabine for 3 days prior to immunotherapy. In one embodiment, the subject has a high tumor burden (as assessed by SPD and/or tumor metabolic volume) when the baseline tumor burden (SPD) is greater than 2500, 3000, 3500, or 4000, preferably greater than 3000 mm² and/or the tumor metabolic volume is above the median for a representative tumor population (e.g., above 100, or above 150 ml).

In one embodiment, the disclosure provides a method of treating a subject with a high international prognostic index, wherein the immune activation mediated stress in the subject is reduced by administering one or more agents or treatments that result in a reduced inflammation (e.g., lower cytokine induction in the blood) and/or by using an alternative lymphodepleting regimen that does not comprise the administration of 500-600 mg/m²/day of cyclophosphamide and 30 mg/m²/day of fludarabine for 3 days prior to immunotherapy. In one embodiment, the subject has a high international prognostic index (IPI) when the IPI is greater than 1, 2 or 3.

In one embodiment, the immunotherapy is T cell therapy. In one embodiment, the T cell therapy is autologous. In one embodiment, the T cell therapy is allogeneic. In some embodiments, the T cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), iPSCs, checkpoint inhibitors, and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells. In one embodiment, the immunotherapy is CAR T cell or TCR T cell therapy. In one embodiment, the immunotherapy is anti-CD19 CAR T cell therapy. Examples of target tumor antigens are listed elsewhere in the specification. Examples of cancers that may be treated by the methods of the disclosure are also provided elsewhere in the specification.

In one embodiment, the agent(s) that is administered in combination with immunotherapy and reduces immune activation and/or endothelial cells disruption, wherein the combination therapy reduces cytokine induction and/or wherein the combination therapy reduces the endothelial cell disruption, is/are selected from anti-IL-1 (e.g. anakinra), T cell activation inhibitors (e.g. dasatinib), JAK inhibitors (e.g. filgotinib), anti-GM-CSF (e.g. lenzilumab), anti-TNF (e.g. infliximab), Ang2 inhibitors (e.g. azilsartan), anti-angiogenic therapies (e.g. bevacizumab), anti-IFNg (e.g. emapalumab-lzsg) etc. In one embodiment, the immunotherapy is administered in a combination therapy that enhances the proliferation of the T cells. In one embodiment, said combination therapy comprises treatment with pembrolizumab, lenalidomide, epcoritamab, and utoliumab. In one embodiment, said therapy comprises magrolimab (anti-CD47 antagonist), GSK3745417 (STING agonist), INCB001158 (ARG1/2 inhibitor), GS-1423 (CD73xTGFβ mAb), Selicrelumab (CD40 agonist), GS3583 (FLT3 agonist), Pexidartinib (CSF1R inhibitor, epacadostat (IDO1 inhibitor), GS9620 (TLR agonist). In one embodiment, the agent is selected from (i) a GM-CSF inhibitor selected from lenzilumab; namilumab (AMG203); GSK3196165/MOR103/otilimab (GSK/MorphoSys); KB002 and KB003 (KaloBios); MT203 (Micromet and Nycomed); MORAb-022/gimsilumab (Morphotek); or a biosimilar of any one of the same; E21R; and a small molecule; (ii) a CSF1 inhibitor selected from RG7155, PD-0360324, MCS110/lacnotuzumab), or a biosimilar version of any one of the same; and a small molecule; and/or (iii) a GM-CSFR inhibitor and the CSF1R inhibitor selected from Mavrilimumab (formerly CAM-3001; MedImmune, Inc.); cabiralizumab (Five Prime Therapeutics); LY3022855 (IMC-CS4)(Eli Lilly), Emactuzumab, also known as RG7155 or RO5509554; FPA008 (Five Prime/BMS); AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax); a biosimilar version of any one of the same; and a small molecule. In some embodiments, additional treatments may be cytokines (e.g., IL-2, IL-15), stimulating antibodies (e.g., anti-41BB, OX-40), checkpoint blockade (e.g., CTLA4, PD-1), or innate immune stimulators (e.g., TLR, STING agonists). In some embodiments, additional treatments may be T cell-recruiting chemokines (e.g., CCL2, CCL1, CCL22, CCL17, and combinations thereof). In some embodiments, the additional therapy or therapies are administered systemically or intratumorally. In some embodiments, the additional therapy that is used in combination is administered together with conditioning and/or immunotherapy. In some embodiments, the additional therapy that is used in combination is administered sequentially with conditioning and/or immunotherapy.

In one embodiment, the agents may/should be administered to the patient prior to, after, and/or during immunotherapy administration to reduce Grade 3+ CRS in the subject. In one embodiment, the agent(s) is/are administered to the patient prior to CAR-T infusion, before the peak of CAR-T expansion (e.g., Day 0-6 post infusion), and/or at the peak CAR-T expansion (e.g., Day 7-14). In one embodiment, the peak of CAR-T expansion is Day 7-14 post infusion. In one embodiment, the peak of CAR-T expansion is Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, or Day 20 post-infusion. In one embodiment, the period after peak CAR-T expansion is the period between Day 14-28 post-infusion. In one embodiment, the period after peak CAR-T expansion is Day 1-Day 5, Day 5-Day 10, Day 10-Day 15, Day 15-Day 20, Day 20-Day 25; after Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, Day 20, Day 25, Day 30, Day 35, Day 40, Day 45, Day 50, any day after peak expansion.

In one embodiment, the immunotherapy is combined with low dose radiation, promotion of T cell activity through immune checkpoint blockade, and/or T cell agonists. In one embodiment, the T cell agonist is selected from pembrolizumab, lenalidomide, epcoritamab, and utoliumab. In one embodiment, the combination agent is selected from check-point inhibitors (e.g., anti-PD1 antibodies, pembrolizumab (Keytruda), Cemiplimab (Libtayo), nivolumab (Opdivo); anti-PD-L1 antibodies, Atezolizumab (Tecentriq), Avelumab (Bavencio), Durvalumab (Imfinzi); and/or anti-CTLA-4 antibodies, Ipilimumab (Yervoy)).

In one embodiment, the pre-conditioning regimen is a lymphodepleting regimen. In one embodiment, the lymphodepletion therapy regimen(s) is/are selected from one of several possible regimens of cyclophosphamide/fludarabine, bendamustine, total body irradiation, Anti-CD45 (Apamistamab), and other chemotherapeutic agents (e.g., AVM0703, Busulfan, Thiotepa/Etoposide, Pentostatin). Additional conditioning methods and regimens can be found elsewhere in the specification.

In one embodiment, the disclosure provides a method of improving immunotherapy (e.g., CAR T cell treatment) by optimization of bridging therapy to modulate the tumor microenvironment to a more favorable immune permissive state. In one embodiment, the optimization comprises administering bridging therapy with Immunomodulatory imide drugs (IMIDs)/cereblon modulators (e.g., lenoalidomide, pomalidomide, iberdomide, and apremilast). In one embodiment, the optimization comprises administering bridging therapy with local radiation.

In one embodiment, the disclosure provides a method of improving immunotherapy (e.g., CAR T cell treatment) by optimization of bridging therapy to diminish tumor burden prior immunotherapy (e.g., CAR T cell treatment) administration. In one embodiment, the optimization comprises administering bridging therapy with R-CHOP, bendamustine, alkylating agents, and/or platinum-based agents. Other exemplary bridging therapies are described elsewhere in this application.

In one embodiment, the disclosure provides a method of improving immunotherapy (e.g., CAR T cell treatment) by optimization of conditioning treatment to modulate the tumor microenvironment to a more favorable immune permissive state (e.g., less myeloid inflammation in the TME). In one embodiment, the optimization comprises addition of local irradiation to cyclophosphamide/fludarabine conditioning. In one embodiment, the optimization comprises administration of platinum-based agents as conditioning agents.

In one embodiment, the disclosure provides a method of improving immunotherapy (e.g., CAR T cell treatment) by coadministration of biological response modifiers together or post-immunotherapy (e.g., CAR T cell treatment) administration to enable CAR T cell activity. In one embodiment, the method comprises administration of gamma chain cytokines (e.g., IL-2, IL-4, IL-7, IL-9, IL-15, and IL-21). In one embodiment, the method comprises administration of checkpoint blocking agents (e.g., anti-CTLA-4).

In one embodiment, the disclosure provides a method of improving immunotherapy (e.g., CAR T cell treatment) by reprogramming of T cells to overcome detrimental tumor microenvironments, including low T/M ratio, high tumor burden, high TME myeloid cell density and/or high TME myeloid inflammation levels. In one embodiment, the T cells are engineered to express gamma chain receptor cytokines. In one embodiment, the gamma chain receptor cytokines are expressed under constitutive or inducible promoters.

In one embodiment, the disclosure provides a method of improving CAR T cell treatment by optimizing T cell manufacturing to help CAR T cells overcome detrimental tumor microenvironments, wherein the characteristics of the tumor microenvironment that may be detrimental comprise low T/M ratio, high tumor burden, high TME myeloid cell density and/or high TME myeloid inflammation levels. In one embodiment, the characteristics of the TME that may be detrimental comprise low T/M ratio (within −0.5-4), high tumor burden (within 3000-40000 mm²), high myeloid cell density (within 1000-4000 cells/mm²) and/or high TME myeloid inflammation levels (within 27-2000). In one embodiment, the method comprises engineering CAR T cells to express gamma chain receptor cytokines. In one embodiment, the gamma chain receptor cytokines are expressed under constitutive or inducible promoters. In one embodiment, the method comprises growing the T cells in the presence of gamma chain cytokines such as IL-15.

### Clinical Outcomes

In some embodiments, the clinical outcome is complete response. In some embodiments, the clinical outcome is durable response. In some embodiments, the clinical outcome is no response. In some embodiments, the clinical outcome is partial response. In some embodiments, the clinical outcome is objective response. In some embodiments, the clinical outcome is survival. In some embodiments, the clinical outcome is relapse.

In some embodiments, objective response (OR) is determined per the revised IWG Response Criteria for Malignant Lymphoma (Cheson, 2007) and determined by IWG Response Criteria for Malignant Lymphoma (Cheson et al. Journal of Clinical Oncology 32, no. 27 (September 2014) 3059-3067). Duration of Response is assessed. The Progression-Free Survival (PFS) by investigator assessment per Lugano Response Classification Criteria is evaluated.

In some embodiments, part of the clinical outcome is the evaluation of adverse events. In this regard, CRS grading was done according to Lee D W et al., (2014). Current concepts in the diagnosis and management of cytokine release syndrome. Blood. 2014 Jul. 10; 124(2): 188-195. Neurologic toxicity was assessed by monitoring patients for signs and symptoms of neurologic toxicities by ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. In some embodiments, the symptom of neurologic toxicity is selected from encephalopathy, headache, tremor, dizziness, aphasia, delirium, insomnia, and anxiety.

In some embodiments, the method comprises monitoring patients at least daily for 7 days at the certified healthcare facility following infusion for signs and symptoms of neurologic toxicities. In some embodiments, the method comprises monitoring patients for signs or symptoms of neurologic toxicities for 4 weeks after infusion.

In some embodiments, the symptom of neurologic toxicity is selected from encephalopathy, headache, tremor, dizziness, aphasia, delirium, insomnia, and anxiety. In some embodiments, the symptom of adverse reaction is selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia. In some embodiments, patients are instructed to remain within proximity of the certified healthcare facility for at least 4 weeks following infusion.

Clinical outcomes of CAR T cell treatment are dependent on the level of CAR T cells in the blood. In some embodiments, response, levels of CAR T cells in blood, or immune related factors is determined by follow up at about 1 day, about 2 days, about 3 days, about 4 days, about 5 days, about 6 days, or about 7 days after administration of engineered CAR T cells. In some embodiments, response, levels of CAR T cells in blood, or immune related factors is determined by follow up at about 1 week, about 2 weeks, about 3 weeks, or about 4 weeks after administration of engineered CAR T cells. In some embodiments, response, levels of CAR T cells in blood and/or immune related factors are determined by follow up at about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months after administration of a engineered CAR T cells. In some embodiments, response, levels of CAR T cells in blood and/or immune related factors are determined by follow up at about 1 year, about 1.5 years, about 2 years, about 2.5 years, about 3 years, about 4 years, or about 5 years after administration of engineered CAR T cells.

In some embodiments, methods described herein may provide a clinical benefit to a subject. In some embodiments, at least 1%, 2%, 3%, 4%, 5%, 6%, 7%, 8%, 9%, 10%, 15%, 20%, 25%, 30%, 35%, 40%, 50%, 55%, 60%, 65%, 70%, 75%, 80%, 85%, 90% or 95% of patients achieve a clinical benefit. In some embodiments, approximately 1%, 2%, 3%, 4%, 5%, 6%, 7%, 8%, 9%, 10%, 15%, 20%, 25%, 30%, 35%, 40%, 45%, 0%, 55%, 60%, 65%, 70%, 75%, 80%, 85%, 90% or 95% and any unenumerated % in between of patients achieve a clinical benefit. In some embodiments, the response rate is 1%, 2%, 3%, 4%, 5%, 6%, 7%, 8%, 9%, 9.5%, 10.5%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 25 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81, 82, 83, 84, 85, 86, 87, 88, 89, 90, 91, 92, 93, 94, 95, 96, 97, 98, 99, or 100% or some other unenumerated percentage and range in between 1% and 100%. In some embodiments, the response rate is between 0%-10%, 10%-20%, 20%-30%, 30%-40%, 40%-50%, 50%-60%, 60%-70%, 70%-80%, 80%-90%, or 90%-100%. In some embodiments, the response rate is between 0%-1.%, 1%-1.5%, 1.5%-2%, 2%-3%, 3%-4%, 4%-5%, 5%-6%, 6%-7%, 7%-8%, 8%-9%, 9%-10%, 10%-15%, 15%-20%, 20-25%, 25%-30%, 35-40%, and so one and so forth, through 95%-100%.

### Chimeric Antigen Receptors

In one embodiment, the immunotherapy is CAR-T cell immunotherapy. Chimeric antigen receptors (CARs) are genetically engineered receptors. These engineered receptors may be inserted into and expressed by immune cells, including T cells and other lymphocytes in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci. Transl. Med. 3:95 (2011); Porter et al., N. Engl. J. Med. 365:725-33 (2011), and Gross et al., Annu. Rev. Pharmacol. Toxicol. 56:59-83 (2016).

In some embodiments, a costimulatory domain which includes a truncated hinge domain (“THD”) further comprises some or all of a member of the immunoglobulin family such as IgG1, IgG2, IgG3, IgG4, IgA, IgD, IgE, IgM, or fragment thereof.

In some embodiments, the THD is derived from a human complete hinge domain (“CHD”). In other embodiments, the THD is derived from a rodent, murine, or primate (e.g., non-human primate) CHD of a costimulatory protein. In some embodiments, the THD is derived from a chimeric CHD of a costimulatory protein.

The costimulatory domain for the CAR of the disclosure may further comprise a transmembrane domain and/or an intracellular signaling domain. The transmembrane domain may be fused to the extracellular domain of the CAR. The costimulatory domain may similarly be fused to the intracellular domain of the CAR. In some embodiments, the transmembrane domain that naturally is associated with one of the domains in a CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this disclosure may be derived from (i.e., comprise) 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

Optionally, short linkers may form linkages between any or some of the extracellular, transmembrane, and intracellular domains of the CAR. The linkers described herein, may also be used as a peptide tag. The linker peptide sequence may be of any appropriate length to connect one or more proteins of interest and is preferably designed to be sufficiently flexible so as to allow the proper folding and/or function and/or activity of one or both of the peptides it connects. Thus, the linker peptide may have a length of no more than 10, no more than 11, no more than 12, no more than 13, no more than 14, no more than 15, no more than 16, no more than 17, no more than 18, no more than 19, or no more than 20 amino acids. In some embodiments, the linker peptide comprises a length of at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids. In some embodiments, the linker comprises at least 7 and no more than 20 amino acids, at least 7 and no more than 19 amino acids, at least 7 and no more than 18 amino acids, at least 7 and no more than 17 amino acids, at least 7 and no more than 16 amino acids, at least 7 and no more 15 amino acids, at least 7 and no more than 14 amino acids, at least 7 and no more than 13 amino acids, at least 7 and no more than 12 amino acids or at least 7 and no more than 11 amino acids. In certain embodiments, the linker comprises 15-17 amino acids, and in particular embodiments, comprises 16 amino acids. In some embodiments, the linker comprises 10-20 amino acids. In some embodiments, the linker comprises 14-19 amino acids. In some embodiments, the linker comprises 15-17 amino acids. In some embodiments, the linker comprises 15-16 amino acids. In some embodiments, the linker comprises 16 amino acids. In some embodiments, the linker comprises 3, 4, 5, 6, 7, 8, 9, 10, 11, 12, 13, 14, 15, 16, 17, 18, 19 or 20 amino acids.

In some embodiments, a spacer domain is used. In some embodiments, the spacer domain is derived from CD4, CD8a, CD8b, CD28, CD28T, 4-1BB, or other molecule described herein. In some embodiments, the spacer domains may include a chemically induced dimerizer to control expression upon addition of a small molecule. In some embodiments, a spacer is not used.

The intracellular (signaling) domain of the engineered T cells of the disclosure may provide signaling to an activating domain, which then activates at least one of the normal effector functions of the immune cell. Effector function of a T cell, for example, may be cytolytic activity or helper activity including the secretion of cytokines.

In certain embodiments, suitable intracellular signaling domain include (i.e., comprise), but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Ly108), lymphocyte function-associated antigen-1 (LFA-1; CD11a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

### Antigen Binding Molecules

Suitable CARs and TCRs may bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (“scFv”). A scFv is a single chain antibody fragment having the variable regions of the heavy and light chains of an antibody linked together. See U.S. Pat. Nos. 7,741,465 and 6,319,494, as well as Eshhar et al., Cancer Immunol Immunotherapy (1997) 45: 131-136. A scFv retains the parent antibody's ability to interact specifically with target antigen. scFv's are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR or TCR such that it is capable of recognizing and binding to the antigen of interest. Bispecific and multispecific CARs and TCRs are contemplated within the scope of the disclosure, with specificity to more than one target of interest.

In some embodiments, the polynucleotide encodes a CAR or TCR comprising a (truncated) hinge domain and an antigen binding molecule that specifically binds to a target antigen. In some embodiments, the target antigen is a tumor antigen. In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), as well as any derivate or variant of these surface antigens.

Engineered T cells and Products

In one embodiment, the immunotherapy is T cell therapy. In some embodiments, the donor T cells for use in the T cell therapy are obtained from the patient (e.g., for an autologous T cell therapy). In other embodiments, the donor T cells for use in the T cell therapy are obtained from a subject that is not the patient. In certain embodiments, the T cell is a tumor-infiltrating lymphocyte (TIL), engineered autologous T cell (eACT™), an allogeneic T cell, a heterologous T cell, or any combination thereof. In some embodiments, the T cells are obtained from a donor subject. In some embodiments, the donor subject is human patient afflicted with a cancer or a tumor. In some embodiments, the donor subject is a human patient not afflicted with a cancer or a tumor.

In one embodiment, the cells are obtained from a subject. In one embodiment, the cells are Induced Pluripotent Stem Cells (iPSCs). T cells may be obtained from, e.g., peripheral blood mononuclear cells, bone marrow, lymph node tissue, cord blood, thymus tissue, tissue from a site of infection, ascites, pleural effusion, spleen tissue, tumors, or differentiated in vitro. In addition, the T cells may be derived from one or more T cell lines available in the art. T cells may also be obtained from a unit of blood collected from a subject using any number of techniques known to the skilled artisan, such as FICOLL™ separation and/or apheresis. In some embodiments, the cells collected by apheresis are washed to remove the plasma fraction and placed in an appropriate buffer or media for subsequent processing. In some embodiments, the cells are washed with PBS. As will be appreciated, a washing step may be used, such as by using a semi-automated flow through centrifuge, e.g., the Cobe™ 2991 cell processor, the Baxter CytoMate™, or the like. In some embodiments, the washed cells are resuspended in one or more biocompatible buffers, or other saline solution with or without buffer. In some embodiments, the undesired components of the apheresis sample are removed. Additional methods of isolating T cells for a T cell therapy are disclosed in U.S. Patent Pub. No. 2013/0287748, which is herein incorporated by references in its entirety.

In some embodiments, T cells are isolated from PBMCs by lysing the red blood cells and depleting the monocytes, e.g., by using centrifugation through a PERCOLL™ gradient. In some embodiments, a specific subpopulation of T cells, such as CD4+, CD8+, CD28+, CD45RA+, and CD45RO+ T cells is further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In some embodiments, PBMCs are used directly for genetic modification with the immune cells (such as CARs) using methods as described herein. In some embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8+ cells are further sorted into naive, central memory, and effector cells by identifying cell surface antigens that are associated with each of these types of CD8+ cells. In some embodiments, the expression of phenotypic markers of central memory T cells includes expression of CCR7, CD3, CD28, CD45RO, CD62L, and CD127 and negative for granzyme B. In some embodiments, central memory T cells are CD8+, CD45RO+, and CD62L+ T cells. In some embodiments, effector T cells are negative for CCR7, CD28, CD62L, and CD127 and positive for granzyme B and perform. In some embodiments, CD4+ T cells are further sorted into subpopulations. For example, CD4+ T helper cells may be sorted into naive, central memory, and effector cells by identifying cell populations that have cell surface antigens.

In some embodiments, the immune cells, e.g., T cells, are genetically modified (engineered) following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, by way of non-limiting example, in U.S. Pat. Nos. 6,905,874, 6,867,041, and 6,797,514, and in International Publication Nos. WO 2015/20096, WO 2016/191756, WO 2016/191755, WO 2019/079564, and WO 2021/092290, each of which are herein incorporated by reference in their entirety. the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is the Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In some embodiments, a composition comprising engineered T cells comprises a pharmaceutically acceptable carrier, diluent, solubilizer, emulsifier, preservative and/or adjuvant. In some embodiments, the composition comprises an excipient.

In some embodiments, the composition is selected for parenteral delivery, for inhalation, or for delivery through the digestive tract, such as orally. The preparation of such pharmaceutically acceptable compositions is within the ability of one skilled in the art. In some embodiments, buffers are used to maintain the composition at physiological pH or at a slightly lower pH, typically within a pH range of from about 5 to about 8. In some embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In some embodiments, the vehicle for parenteral injection is sterile distilled water in which composition described herein, with or without at least one additional therapeutic agent, is formulated as a sterile, isotonic solution, properly preserved. In some embodiments, the preparation involves the formulation of the desired molecule with polymeric compounds (such as polylactic acid or polyglycolic acid), beads or liposomes, that provide for the controlled or sustained release of the product, which are then be delivered via a depot injection. In some embodiments, implantable drug delivery devices are used to introduce the desired molecule.

In some embodiments, the engineered T cells are administered at a therapeutically effective amount. For example, a therapeutically effective amount of the engineered T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, the therapeutically effective amount of the engineered viable T cells is between about 1×10⁶ and about 2×10⁶ engineered viable T cells per kg body weight up to a maximum dose of about 1×10⁸ engineered viable T cells.

In some embodiments, the engineered T cells are anti-CD19 CART T cells. In some embodiments, the anti-CD19 CAR T cells are the axicabtagene ciloleucel product, YESCARTA™ axicabtagene ciloleucel (axi-cel), TECARTUS™—brexucabtagene autoleucel/KTE-X19, KYMRIAH™ (tisagenlecleucel), lisocabtagene maraleucel, In some embodiments, the engineered T cells are anti-BCMA CAR T cells, such as Idecabtagene vicleucel/bb2121 etc. In some embodiments, the product meets commercial specifications. In some embodiments, the product does not meet commercial specifications (out-of-specification product, OOS). In some embodiments, the OOS product comprises fewer, less differentiated CCR7+ T_{N} and T_{CM} and a greater proportion of more differentiated CCR7− T_{EM}+T_{EFF} cells than the axicabtagene ciloleucel product that meets commercial specifications. In some embodiments, the OOS product results in a median peak CAR T cell level after administration that is lower than that of the commercial product. In some embodiments, the OOS product still showed a manageable safety profile and meaningful clinical benefit.

The application also provides dosages and administrations of cells prepared by the methods of the application, for example, an infusion bag of CD19-directed genetically modified autologous T cell immunotherapy, comprises a suspension of chimeric antigen receptor (CAR)-positive T cells in approximately 68 mL for infusion. In some embodiments, the CAR T cells are formulated in approximately 40 mL for infusion In some embodiments, the CAR T cell product is formulated in a total volume of 35, 40, 45, 50, 55, 60, 65, 70, 75, 80, 85, 90, 95, 100, 200, 300, 400, 500, 500, 700, 800, 900, 1000 mL. In one aspect, the dosage and administration of cells prepared by the methods of the application, for example, an infusion bag of CD19-directed genetically modified autologous T cell immunotherapy, comprises a suspension of 1×10⁶ CAR-T positive cells in approximately 40 mL. The target dose may be between about 1×10⁶ and about 2×10⁶ CAR-positive viable T cells per kg body weight, with a maximum of 2×10⁸ CAR-positive viable T cells.

In some embodiments, the dosage form comprises a cell suspension for infusion in a single-use, patient-specific infusion bag; the route of administration is intravenous; the entire contents of each single-use, patient-specific bag is infused by gravity or a peristaltic pump over 30 minutes. In one embodiment, the dosing regimen is a single infusion consisting of 2.0×10⁶ anti-CD19 CAR T cells/kg of body weight (±20%), with a maximum dose of 2×10⁸ anti-CD19 CAR T cells (for subjects ≥100 kg). In some embodiments, the T cells that make up the dose are CD19 CAR-T cells.

### Conditioning Agents

In some embodiments, the subject is administered a conditioning agent prior to immunotherapy. In some embodiments, conditioning is done with radiation treatment. In some embodiments, the conditioning therapy is a lymphodepleting chemotherapy.

In one embodiment, the conditioning therapy comprises an alkylating agent selected from the group consisting of melphalan, chlorambucil, cyclophosphamide, mechlorethamine, mustine (HN2), uramustine, uracil mustard, melphalan, chlorambucil, ifosfamide, bendamustine, carmustine, lomustine, streptozocin, alkyl sulfonates, busulfan, thiotepa or its analogues, and any combination thereof; a purine analogs selected from the group consisting of azathioprine, 6-mercaptopurine, mercaptopurine, thiopurines, thioguanine, fludarabine, pentostatin, cladribine, and any combination thereof; and/or a platinum-based preconditioning agents selected from the group consisting of platinum, cisplatin, carboplatin, nedaplatin, oxaliplatin, satraplatin, triplatin tetranitrate, procarbazine, altretamine, triazenes, dacarbazine, mitozolomide, temozolomide, dacarbazine, temozolomide, and any combination thereof.

In another embodiment, the one or more preconditioning agents can include platinum-based chemotherapeutic agents. In certain embodiments, the platinum-based chemotherapeutic agents are selected from the group consisting of platinum, cisplatin, carboplatin, nedaplatin, oxaliplatin, satraplatin, triplatin tetranitrate, procarbazine, altretamine, triazenes, dacarbazine, mitozolomide, temozolomide, dacarbazine, temozolomide, any analogues or functional derivatives thereof, and any combination thereof.

In another embodiment, the one or more preconditioning agents can include purine analogues. In certain embodiments, the purine analogues are selected from the group consisting of azathioprine, 6-mercaptopurine, mercaptopurine, thiopurines, thioguanine, fludarabine, pentostatin, cladribine, any analogue or functional derivative thereof, and any combination thereof. In one embodiment, the one or more preconditioning agents includes fludarabine.

In some embodiments, the one or more preconditioning agents can include cyclophosphamide and a purine analog. The purine analogues can be selected from the group consisting of azathioprine, 6-mercaptopurine, mercaptopurine, thiopurines, thioguanine, fludarabine, pentostatin, cladribine, any analogue or functional derivative thereof, and any combination thereof. In one particular embodiment, the one or more preconditioning agents include cyclophosphamide and pentostatin. In one particular embodiment, the one or more preconditioning agents include cyclophosphamide and fludarabine. By way of non-limiting example, dosing amounts and regimens of cyclophosphamide and fludarabine are described in at least International Publication No. WO 2019/079564, International Publication No. WO 2021/092290, International Publication No. WO 2015/20096, and International Publication No. WO 2016/191755 each of which are herein incorporated by reference in their entirety.

In certain embodiments, a first dose (also applies to repeated doses) of the one or more preconditioning agents is administered to the patient. For example, in some embodiments, a first dose of cyclophosphamide is about 300 mg/m²/day to about 2000 mg/m²/day. In another embodiment, the first dose of cyclophosphamide is higher than 300 mg/m²/day and lower than 2000 mg/m²/day. In other embodiments, the dose of cyclophosphamide is about 350 mg/m²/day-about 2000 mg/m²/day, at least about 400 mg/m²/day-about 2000 mg/m²/day, about 450 mg/m²/day-about 2000 mg/m²/day, about 500 mg/m²/day-about 2000 mg/m²/day, about 550 mg/m²/day-about 2000 mg/m²/day, or about 600 mg/m²/day-about 2000 mg/m²/day. In other embodiments, the dose of cyclophosphamide is about 350 mg/m²/day-about 1500 mg/m²/day, about 350 mg/m²/day-about 1000 mg/m²/day, about 400 mg/m²/day-about 900 mg/m²/day, about 450 mg/m²/day-about 800 mg/m²/day, about 450 mg/m²/day-about 700 mg/m²/day, about 500 mg/m²/day-about 600 mg/m²/day, or about 300 mg/m²/day-about 500 mg/m²/day. In another embodiment, the dose of cyclophosphamide is about 350 mg/m²/day, about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, or about 1000 mg/m²/day.

In other embodiments, the first dose (also applies to repeated doses) of cyclophosphamide is about 200 mg/m²/day to about 3000 mg/m²/day. In another embodiment, the first dose of cyclophosphamide is higher than 200 mg/m²/day and lower than 3000 mg/m²/day. In other embodiments, the dose of cyclophosphamide is about 200 mg/m²/day-about 3000 mg/m²/day, about 300 mg/m²/day-about 3000 mg/m²/day, about 400 mg/m²/day-about 3000 mg/m²/day, about 500 mg/m²/day-about 3000 mg/m²/day, about 600 mg/m²/day-about 3000 mg/m²/day, about 700 mg/m²/day-about 3000 mg/m²/day, about 800 mg/m²/day-about 3000 mg/m²/day, about 900 mg/m²/day-about 3000 mg/m²/day, about 1000 mg/m²/day-about 3000 mg/m²/day, about 1100 mg/m²/day-about 3000 mg/m²/day, about 1200 mg/m²/day-about 3000 mg/m²/day, about 1300 mg/m²/day-about 3000 mg/m²/day, about 1400 mg/m²/day-about 3000 mg/m²/day, about 1500 mg/m²/day-about 3000 mg/m²/day, about 1600 mg/m²/day-about 3000 mg/m²/day, about 1700 mg/m²/day-about 3000 mg/m²/day, about 1800 mg/m²/day-about 3000 mg/m²/day, about 1900 mg/m²/day-about 3000 mg/m²/day, about 2000 mg/m²/day-about 3000 mg/m²/day, about 200 mg/m²/day-about 2900 mg/m²/day, about 400 mg/m²/day-about 2800 mg/m²/day, about 500 mg/m²/day-about 2700 mg/m²/day, about 600 mg/m²/day-about 2600 mg/m²/day, about 700 mg/m²/day-about 2500 mg/m²/day, about 800 mg/m²/day-about 2400 mg/m²/day, about 900 mg/m²/day-about 2350 mg/m²/day, about 1000 mg/m²/day-about 2300 mg/m²/day, about 1100 mg/m²/day-about 2250 mg/m²/day, or about 1110 mg/m²/day-about 2220 mg/m²/day. In one embodiment, the first dose of cyclophosphamide is 200 mg/m²/day. In another embodiment, the first dose of cyclophosphamide is 300 mg/m²/day. In another embodiment, the first dose of cyclophosphamide is 500 mg/m²/day.

In some embodiments, a first dose (also applies to repeated doses) of fludarabine is about 20 mg/m²/day to about 900 mg/m²/day. In some embodiments, a dose of fludarabine is higher than 30 mg/m²/day and lower than 900 mg/m²/day. In some embodiments, a dose fludarabine is about 35 mg/m²/day-about 900 mg/m²/day, about 40 mg/m²/day-about 900 mg/m²/day, about 45 mg/m²/day-about 900 mg/m²/day, about 50 mg/m²/day-about 900 mg/m²/day, about 55 mg/m²/day-about 900 mg/m²/day, or about 60 mg/m²/day-about 900 mg/m²/day. In some embodiments, a dose of fludarabine is about 35 mg/m²/day-about 900 mg/m²/day, about 35 mg/m²/day-about 800 mg/m²/day, about 35 mg/m²/day-about 700 mg/m²/day, about 35 mg/m²/day-about 600 mg/m²/day, about 35 mg/m²/day-about 500 mg/m²/day, about 35 mg/m²/day-about 400 mg/m²/day, about 35 mg/m²/day-about 300 mg/m²/day, about 35 mg/m²/day-about 200 mg/m²/day, about 35 mg/m²/day-about 100 mg/m²/day, about 40 mg/m²/day-about 90 mg/m²/day, about 45 mg/m²/day-about 80 mg/m²/day, about 45 mg/m²/day-about 70 mg/m²/day, or about 50 mg/m²/day-about 60 mg/m²/day. In some embodiments, a dose of fludarabine is about 20 mg/m²/day, about 25 mg/m²/day, about 30 mg/m²/day, about 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, about 100 mg/m²/day, about 200 mg/m²/day, or about 300 mg/m²/day. In some embodiments, a dose of fludarabine is about 20 mg/m²/day, about 25 mg/m²/day, about 30 mg/m²/day, about 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, or about 100 mg/m²/day. In other embodiments, the dose of fludarabine is about 110 mg/m²/day, 120 mg/m²/day, 130 mg/m²/day, 140 mg/m²/day, 150 mg/m²/day, 160 mg/m²/day, 170 mg/m²/day, 180 mg/m²/day, or 190 mg/m²/day. In some embodiments, the dose of fludarabine is about 210 mg/m²/day, 220 mg/m²/day, 230 mg/m²/day, 240 mg/m²/day, 250 mg/m²/day, 260 mg/m²/day, 270 mg/m²/day, 280 mg/m²/day, or 290 mg/m²/day. In one particular embodiment, the dose of fludarabine is about 20 mg/m²/day. In one particular embodiment, the dose of fludarabine is about 25 mg/m²/day. In another embodiment, dose of fludarabine is about 30 mg/m²/day. In another embodiment, dose of fludarabine is about 60 mg/m²/day.

The timing of the administration of the one or more preconditioning agents can be adjusted to maximize effect. In certain embodiments, the one or more preconditioning agents comprise at two or more preconditioning agents. The two or more preconditioning agents can be administered concurrently or sequentially. In one particular embodiment, a first preconditioning agent, e.g., cyclophosphamide, is administered to the patient prior to or after a second preconditioning agent, e.g., fludarabine.

The doses of cyclophosphamide and fludarabine can be raised or lowered together or independently. For example, the dose of cyclophosphamide can be increased while the dose of fludarabine is decreased, and the dose of cyclophosphamide can be decreased while the dose of fludarabine is increased. Alternatively, the dose of both cyclophosphamide and fludarabine can be increased or decreased together. In some embodiments, the dose of cyclophosphamide is 300 mg/m²/day and the dose of fludarabine is 20 mg/m²/day. In other embodiments, the dose of cyclophosphamide is 300 mg/m²/day and the dose of fludarabine is 30 mg/m²/day. In other embodiments, the dose of cyclophosphamide is 300 mg/m²/day and the dose of fludarabine is 60 mg/m²/day. In other embodiments, the dose of cyclophosphamide is 500 mg/m²/day and the dose of fludarabine is 20 mg/m²/day. In other embodiments, the dose of cyclophosphamide is 500 mg/m²/day and the dose of fludarabine is 30 mg/m²/day. In other embodiments, the dose of cyclophosphamide is 500 mg/m²/day and the dose of fludarabine is 60 mg/m²/day. In other embodiments, the dose of cyclophosphamide is 200 mg/m²/day and the dose of fludarabine is 20 mg/m²/day. In other embodiments, the dose of cyclophosphamide is 200 mg/m²/day and the dose of fludarabine is 30 mg/m²/day. In other embodiments, the dose of cyclophosphamide is 200 mg/m²/day and the dose of fludarabine is 60 mg/m²/day.

As described herein, the day that a T cell therapy is administered is designated as day 0. The one or more preconditioning agents can be administered at any time prior to administration of the T cell therapy. In some embodiments, the administration of the one or more preconditioning agents begins at least seven days, at least six days, at least five days, at least four days, at least three days, at least two days, or at least one day prior to the administration of the T cell therapy. In other embodiments, the administration of the one or more preconditioning agents begins at least eight days, at least nine days, at least ten days, at least eleven days, at least twelve days, at least thirteen days, or at least fourteen days prior to the administration of the T cell therapy. In one embodiment, the administration of the one or more preconditioning agents begins about seven days prior to the administration of the T cell therapy. In another embodiment, the administration of the one or more preconditioning agents begins about five days prior to the administration of the T cell therapy.

In one embodiment, the administration of a first preconditioning agent begins about seven days prior to the administration of the T cell therapy, and the administration of a second preconditioning agent begins about five days prior to administration of the T cell therapy. In one particular embodiment, a first preconditioning agent is administered to the patient for two days at about seven days and about six days prior to the administration of the T cell therapy. In another embodiment, a second preconditioning agent is administered to the patient for five days at about five, four, three, two, and one day prior to the administration of the T cell therapy. In another embodiment, a first preconditioning agent is administered to the patient for three days at about five, four, and three days prior to the administration of the T cell therapy.

In one particular embodiment, administration of the cyclophosphamide begins about seven days prior to the administration of the T cell therapy, and the administration of a purine analog (e.g., fludarabine or pentostatin) begins about five days prior to the administration of the T cell therapy. In another embodiment, administration of the cyclophosphamide begins about five days prior to the administration of the T cell therapy, and the administration of a purine analog (e.g., fludarabine or pentostatin) begins about five days prior to the administration of the T cell therapy.

The timing of the administration of each component can be adjusted to maximize effect. In general, the one or more preconditioning agents can be administered daily. In some embodiments, the one or more preconditioning agents are administered daily for about two days, for about three days, for about four days, for about five days, for about six days, or for about seven days. In some embodiments, the one or more preconditioning agents can be administered daily for at least one day, at least two days, at least three days, at least four days, at least five days, at least six days, or at least seven days. In one particular embodiment, the one or more preconditioning agents are administered daily for about three days.

As described herein, the day the T cell therapy is administered to the patient is designated as day 0. In some embodiments, the one or more preconditioning agents, e.g., the cyclophosphamide, is administered to the patient on day 7 and day 6 prior to day 0 (i.e., day −7 and day −6). In other embodiments, the one or more preconditioning agents, e.g., the cyclophosphamide, is administered to the patient on day −5, day −4, and day −3. In some embodiments, the one or more preconditioning agents, e.g., the fludarabine, is administered to the patient on day −5, day −4, day −3, day −2, and day −1. In other embodiments, the one or more preconditioning agents, e.g., fludarabine, is administered to the patient on day −5, day −4, and day −3.

The one or more preconditioning agents, e.g., the cyclophosphamide and fludarabine, can be administered on the same or different days. If cyclophosphamide and fludarabine are administered on the same day, the cyclophosphamide dose can be administered either before or after the fludarabine dose. In one embodiment, the cyclophosphamide dose is administered to the patient on day −7 and day −6, and the fludarabine dose is administered to the patient on day −5, day −4, day −3, day −2, and day −1. In another embodiment, the cyclophosphamide dose is administered to the patient on day −5, day −4, and day −3, and the fludarabine dose is administered to the patient on day −5, day −4, and day −3.

In certain embodiments, the one or more preconditioning agents, e.g., cyclophosphamide and fludarabine, can be administered concurrently or sequentially. In one embodiment, cyclophosphamide is administered to the patient prior to fludarabine. In another embodiment, cyclophosphamide is administered to the patient after fludarabine.

Routes and regimes for administrating the one or more preconditioning agents are known in the art, and are described, for example, at least in International Publication No. WO 2019/079564, International Publication No. WO 2021/092290, International Publication No. WO 2015/20096, and International Publication No. WO 2016/191755 each of which are herein incorporated by reference in their entirety.

### Cancers

The methods disclosed herein may be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In some embodiments, the methods induce a complete response. In other embodiments, the methods induce a partial response.

Cancers that may be treated include tumors that are not vascularized, not yet substantially vascularized, or vascularized. The cancer may also include solid or non-solid tumors. In some embodiments, the cancer is a hematologic cancer. In some embodiments, the cancer is of the white blood cells. In other embodiments, the cancer is of the plasma cells. In some embodiments, the cancer is leukemia, lymphoma, or myeloma. In some embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, Waldenstrom macroglobulinemia, DLBCL arising from FL, high grade B cell lymphoma, or a combination thereof.

In some embodiments, the cancer is a myeloma. In some embodiments, the cancer is multiple myeloma. In some embodiments, the cancer is leukemia. In some embodiments, the cancer is acute myeloid leukemia. In some embodiments, the cancer is relapsed or refractory large B-cell lymphoma (possibly, after two or more lines of systemic therapy), including diffuse large B-cell lymphoma (DLBCL) not otherwise specified, primary mediastinal large B-cell lymphoma, high grade B-cell lymphoma, and DLBCL arising from follicular lymphoma, or relapsed or refractory follicular lymphoma (FL) (possibly, after two or more lines of systemic therapy), or relapsed or refractory mantle cell lymphoma (MCL).

In some embodiments, the cancer is Non-Hodgkins lymphoma. In some embodiments, the cancer is relapsed/refractory NHL. In some embodiments, the cancer is mantle cell lymphoma.

In some embodiments, the cancer is advanced-stage indolent non-Hodgkin lymphoma (iNHL), including follicular lymphoma (FL) and marginal zone lymphoma (MZL). In some embodiments, the patient has had relapsed/refractory disease after ≥2 prior lines of therapy, including an anti-CD20 monoclonal antibody with an alkylating agent. In some embodiments, the patient may have received a PI3K inhibitor. In some embodiments, the patient may (also) have received autologous stem cell transplantation. In some embodiments, the patient undergoes leukapheresis to obtain T cells for CAR T cell manufacturing, followed by conditioning chemotherapy with cyclophosphamide at 500 mg/m²/day and fludarabine at 30 mg/m²/day administered on days −5, −4, and −3; on day 0, the patient may receive a single intravenous infusion of CAR T cell therapy (e.g., axicabtagene ciloleucel, brexucabtagene autoleucel) at a target dose of 2×10⁶ CAR T cells/kg. In some embodiments, additional infusions may be given at a later period. In some embodiments, if the patient progresses after responding at the month 3 assessment after initial administration, the patient may receive retreatment with CAR T cell treatment (e.g., axicabtagene ciloleucel, brexucabtagene autoleucel). In some embodiments, the patient may receive bridging therapy. Examples of bridging therapies are provided elsewhere in the specification, including the Examples. In some embodiments, the patient experiences CRS. In some embodiments, CRS is managed using any one of the protocols described in this application, including the Examples. In some embodiments, CRS is managed with tocilizumab, corticosteroids and/or vasopressor.

In some embodiments, the cancer is relapsed/refractory indolent Non-Hodgkin Lymphoma and the method of treating a subject in need thereof comprises administering to the subject a therapeutically effective amount of CAR T cells as a retreatment, wherein the subject has previously received a first treatment with CAR T cells. In some embodiments, the first treatment with CAR T cells may have been administered as a first line therapy or a second line therapy, optionally wherein the lymphoma is R/R follicular lymphoma (FL) or marginal zone lymphoma (MZL) and optionally wherein the previous prior lines of therapy included anti-CD20 monoclonal antibody combined with an alkylating agent. In some embodiments, the conditioning therapy comprises fludarabine 30 mg/m² IV and cyclophosphamide 500 mg/m² IV on Days −5, −4, and −3. In some embodiments, the CAR T cell treatment comprises single IV infusion of 2×10⁶ CAR T cells/kg on Day 0. In some embodiments, at least about 10⁴ cells, at least about 10⁵ cells, at least about 10⁶ cells, at least about 10⁷ cells, at least about 10⁸ cells, at least about 10⁹, or at least about 10¹⁰ CAR T cells are administered. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg In some embodiments, the CAR T cells are anti-CD19 CAR T cells. In some embodiments, the CAR T cells are axicabtagene ciloleucel CAR T cells. In some embodiments, the retreatment eligibility criteria include response of a CR or PR at the month 3 disease assessment with subsequent progression; no evidence of CD19 loss in progression biopsy by local review; and/or no Grade 4 CRS or neurologic events, or life-threatening toxicities with the first treatment with CAR T cells. In some embodiments, the method of treatment is that followed by the clinical trial (NCT03105336).

In some embodiments, the cancer is NHL and the immunotherapy (e.g, CAR T or TCR T cell treatment) is administered as a first line therapy. In some embodiments, the cancer is LBCL. In some embodiments, the LBCL is high risk/high grade LBCL with MYC and BCL2 and/or BCL6 translocations or DLBCL with IPI score ≥3 any time before enrollment. In some embodiments, the first line therapy comprises CAR T cell treatment in combination with an anti-CD20 monoclonal antibody and anthracycline-containing regimen. In some embodiments, the CAR T cell treatment is administered first. In some embodiments, the anti-CD20 monoclonal antibody/anthracycline-containing regimen is administered first. In some embodiments, the treatments are administered at least 2 weeks, at least 4 weeks, at least 6 weeks, at least 1 month, at least 2 months, at least 3 months, at least 4 months, at least 5 months, less than a year apart, etc. In some embodiments, the method further comprises bridging therapy administered after leukapheresis and completed prior to initiating conditioning chemotherapy. In some embodiments, additional inclusion criteria include age ≥18 years and ECOG PS 0-1. In some embodiments, the conditioning therapy comprises fludarabine 30 mg/m² IV and cyclophosphamide 500 mg/m² IV on Days −5, −4, and −3. Other exemplary beneficial preconditioning treatment regimens are described in U.S. Provisional Patent Applications 62/262,143 and 62/167,750 and U.S. Pat. Nos. 9,855,298 and 10,322,146, which are hereby incorporated by reference in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient. Another embodiment comprises serum cyclophosphamide and fludarabine at days −4, −3, and −2 prior to T cell administration at a dose of 500 mg/m² of body surface area of cyclophosphamide per day and a dose of 30 mg/m² of body surface area per day of fludarabine during that period of time. Another embodiment comprises cyclophosphamide at day −2 and fludarabine at days −4, −3, and −2 prior to T cell administration, at a dose of 900 mg/m² of body surface area of cyclophosphamide and a dose of 25 mg/m² of body surface area per day of fludarabine during that period of time. In another embodiment, the conditioning comprises cyclophosphamide and fludarabine at days −5, −4 and −3 prior to T cell administration at a dose of 500 mg/m² of body surface area of cyclophosphamide per day and a dose of 30 mg/m² of body surface area of fludarabine per day during that period of time. Other preconditioning embodiments comprise 200-300 mg/m² of body surface area of cyclophosphamide per day and a dose of 20-50 mg/m² of body surface area per day of fludarabine for three days. In some embodiments, the CAR T cell treatment comprises single IV infusion of 2×10⁶ CAR T cells/kg on Day 0. In some embodiments, at least about 10⁴ cells, at least about 10⁵ cells, at least about 10⁶ cells, at least about 10⁷ cells, at least about 10⁸ cells, at least about 10⁹, or at least about 10¹⁰ CAR T cells are administered. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg In some embodiments, the CAR T cells are anti-CD19 CAR T cells. In some embodiments, the CAR T cell treatment comprises anti-CD19 CAR T cells. In some embodiments, the CAR T cell treatment comprises axicabtagene ciloleucel or YESCARTA™. In some embodiments, the CAR T cell treatment comprises TECARTUS™—brexucabtagene autoleucel or KYMRIAH™ (tisagenlecleucel), etc), Idecabtagene vicleucel/bb2121.

In another embodiment, the disclosure provides a method of treating cancer in a subject in need thereof, comprising administering a therapeutically effective amount of CD19 CAR-T treatment to a subject in which the number of lines of prior therapy are 1-2; 3; 4; or ≥5. In one embodiment, the disclosure provides a method of treating cancer in a subject in need thereof, comprising administering a therapeutically effective amount of CD19 CAR-T treatment to a subject in which the number of lines of prior therapy are 1-2. The cancer may be any one of the above listed cancers. The CD19 CAR-T treatment may be any one of the above listed CD19 CAR-T treatments. In some embodiments, the CD19 CAR-T treatment is used as first line of treatment. In some embodiments, the CD19 CAR-T treatment is used as a second line of treatment.

In one embodiment, the CD19 CAR-T treatment is any of the of CD19 CAR-T treatments described above. In one embodiment, the CD19 CAR-T treatment comprises axicabtagene ciloleucel treatment. In embodiments, the cancer is refractory DLBCL not otherwise specified (ABC/GCB), HGBL with or without MYC and BCL2 and/or BCL6 rearrangement, DLBCL arising from FL, T-cell/histiocyte rich large B-cell lymphoma, DLBCL associated with chronic inflammation, Primary cutaneous DLBCL, leg type, and/or Epstein-Barr virus (EBV)+DLBCL. In one embodiment, a subject selected for axicabtagene ciloleucel treatment has refractory DLBCL not otherwise specified (ABC/GCB), HGBL with or without MYC and BCL2 and/or BCL6 rearrangement, DLBCL arising from FL, T-cell/histiocyte rich large B-cell lymphoma, DLBCL associated with chronic inflammation, Primary cutaneous DLBCL, leg type, and/or Epstein-Barr virus (EBV)+DLBCL. In some embodiments, axicabtagene ciloleucel treatment is used as a second line of treatment, where the first line therapy is CHOP, i.e., Cyclophosphamide (Cytoxan®), Doxorubicin (hydroxydoxorubicin), Vincristine (Oncovin®), and Prednisone. In some embodiments, axicabtagene ciloleucel treatment is used as a second line of treatment, where the first line therapy is R-CHOP (CHOP plus Rituximab).

In embodiments, a patient is selected for second-line axicabtagene ciloleucel treatment that has relapsed or refractory disease after first-line chemoimmunotherapy, refractory disease defined as no complete remission to first-line therapy; individuals who are intolerant to first-line therapy are excluded. progressive disease (PD) as best response to first-line therapy, stable disease (SD) as best response after at least 4 cycles of first-line therapy (eg, 4 cycles of R-CHOP), partial response (PR) as best response after at least 6 cycles and biopsy-proven residual disease or disease progression≤12 months of therapy, and/or relapsed disease defined as complete remission to first-line therapy followed by biopsy-proven relapse≤12 months of first-line therapy. In some embodiments, a patient selected for second-line axicabtagene ciloleucel treatment is provided conditioning therapy comprising fludarabine 30 mg/m² IV and cyclophosphamide 500 mg/m² IV on Days −5, −4, and −3. In some embodiments, axicabtagene ciloleucel treatment is used as a second line of treatment.

### Combination Treatments

Compositions comprising CAR-expressing immune effector cells disclosed herein may be administered in conjunction (before, after, and/or concurrently with T cell administration) with any number of chemotherapeutic agents. In some embodiments, the antigen binding molecule, transduced (or otherwise engineered) cells (such as CARs), and the chemotherapeutic agent are administered each in an amount effective to treat the disease or condition in the subject. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylol melamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; Polysaccharide K (PSK); razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. In some embodiments, compositions comprising CAR-expressing immune effector cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone, R-CHOP (CHOP plus Rituximab), and G-CHOP (CHOP plus obinutuzumab).

In some embodiments, the chemotherapeutic agent is administered at the same time or within one week after the administration of the engineered cell. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In some embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein (before, after, and/or concurrently with T cell administration). For example, potentially useful additional therapeutic agents include PD-1 inhibitors such as nivolumab (OPDIVO®), pembrolizumab (KEYTRUDA®), Cemiplimab (Libtayo), pidilizumab (CureTech), and atezolizumab (Roche), and PD-L1 inhibitors such as atezolizumab, durvalumab, and avelumab. In some embodiments, the therapeutic agent(s) to use in combination is anti-IL-1 (e.g., anakinra), T cell activation inhibitors (e.g., dasatinib), JAK inhibitors (e.g., filgotinib), anti-GM-CSF (e.g., lenzilumab), anti-TNF (e.g., infliximab), Ang2 inhibitors (e.g., azilsartan), anti-angiogenic therapies (e.g., bevacizumab), and/or anti-IFNg (e.g., emapalumab-lzsg)

Additional therapeutic agents suitable for use in combination (before, after, and/or concurrently with T cell administration) with the compositions and methods disclosed herein include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib), inhibitors of GM-CSF, CSF1, GM-CSFR, or CSF1R, in addition to anti-thymocyte globulin, lenzilumab and mavrilimumab.

In one embodiment, the GM-CSF inhibitor is selected from lenzilumab; namilumab (AMG203); GSK3196165/MOR103/otilimab (GSK/MorphoSys); KB002 and KB003 (KaloBios); MT203 (Micromet and Nycomed); MORAb-022/gimsilumab (Morphotek); or a biosimilar of any one of the same; E21R; and a small molecule. In one embodiment, the CSF1 inhibitor is selected from RG7155, PD-0360324, MCS110/lacnotuzumab), or a biosimilar version of any one of the same; and a small molecule. In one embodiment, the GM-CSFR inhibitor and the CSF1R inhibitor is/are selected from Mavrilimumab (formerly CAM-3001; MedImmune, Inc.); cabiralizumab (Five Prime Therapeutics); LY3022855 (IMC-CS4)(Eli Lilly), Emactuzumab, also known as RG7155 or R05509554; FPA008 (Five Prime/BMS); AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax); a biosimilar version of any one of the same; and a small molecule.

In some embodiments, a composition comprising an immunotherapy (e.g., engineered CAR T cells) is administered with an anti-inflammatory agent (before, after, and/or concurrently with T cell administration). Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

In some embodiments, the compositions described herein are administered in conjunction with a cytokine (before, after, or concurrently with T cell administration). Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and —II; erythropoietin (EPO, Epogen®, Procrit®); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some embodiments, the administration of the cells and the administration of the additional therapeutic agent are carried out on the same day, are carried out no more than 36 hours apart, no more than 24 hours apart, no more than 12 hours apart, no more than 6 hours apart, no more than 4 hours apart, no more than 2 hours apart, or no more than 1 hour apart or no more than 30 minutes apart. In some embodiments, the administration of the cells and the administration of the additional therapeutic agent are carried out between at or about 0 and at or about 48 hours, between at or about 0 and at or about 36 hours, between at or about 0 and at or about 24 hours, between at or about 0 and at or about 12 hours, between at or about 0 and at or about 6 hours, between at or about 0 and at or about 2 hours, between at or about 0 and at or about 1 hours, between at or about 0 and at or about 30 minutes, between at or about 30 minutes and at or about 48 hours, between at or about 30 minutes and at or about 36 hours, between at or about 30 minutes and at or about 24 hours, between at or about 30 minutes and at or about 12 hours, between at or about 30 minutes and at or about 6 hours, between at or about 30 minutes and at or about 4 hours, between at or about 30 minutes and at or about 2 hours, between at or about 30 minutes and at or about 1 hour, between at or about 1 hours and at or about 48 hours, between at or about 1 hour and at or about 36 hours, between at or about 1 hour and at or about 24 hours, between at or about 1 hour and at or about 12 hours, between at or about 1 hour and at or about 6 hours, between at or about 1 hour and at or about 4 hours, between at or about 1 hour and at or about 2 hours, between at or about 2 hours and at or about 48 hours, between at or about 2 hours and at or about 36 hours, between at or about 2 hours and at or about 24 hours, between at or about 2 hours and at or about 12 hours, between at or about 2 hours and at or about 6 hours, between at or about 2 hours and at or about 4 hours, between at or about 4 hours and at or about 48 hours, between at or about 4 hours and at or about 36 hours, between at or about 4 hours and at or about 24 hours, between at or about 4 hours and at or about 12 hours, between at or about 4 hours and at or about 6 hours, between at or about 6 hours and at or about 48 hours, between at or about 6 hours and at or about 36 hours, between at or about 6 hours and at or about 24 hours, between at or about 6 hours and at or about 12 hours, between at or about 12 hours and at or about 48 hours, between at or about 12 hours and at or about 36 hours, between at or about 12 hours and at or about 24 hours, between at or about 24 hours and at or about 48 hours, between at or about 24 hours and at or about 36 hours or between at or about 36 hours and at or about 48 hours. In some embodiments, the cells and the additional therapeutic agent are administered at the same time.

In some embodiments, the agent is administered in a dosage amount of from or from about 30 mg to 5000 mg, such as 50 mg to 1000 mg, 50 mg to 500 mg, 50 mg to 200 mg, 50 mg to 100 mg, 100 mg to 1000 mg, 100 mg to 500 mg, 100 mg to 200 mg, 200 mg to 1000 mg, 200 mg to 500 mg or 500 mg to 1000 mg.

In some embodiments, the agent is administered in a dosage amount from 0.5 mg/kg to 100 mg/kg, 1 mg/kg to 50 mg/kg, 1 mg kg to 25 mg/kg, 1 mg/kg to 10 mg/kg, 1 mg/kg to 5 mg/kg, 5 mg/kg to 100 mg/kg, 5 mg/kg to 50 mg/kg, 5 mg/kg to 25 mg/kg, 5 mg/kg to 10 mg/kg, 10 mg/kg to 100 mg/kg, 10 mg/kg to 50 mg/kg, 10 mg/kg to 25 mg/kg, 25 mg/kg to 100 mg/kg, 25 mg/kg to 50 mg/kg to 50 mg/kg to 100 mg/kg. In some embodiments, the agent is administered in a dosage amount from 1 mg/kg to 10 mg/kg, 2 mg kg/to 8 mg/kg, 2 mg/kg to 6 mg/kg, 2 mg/kg to 4 mg/kg or 6 mg/kg to 8 mg/kg, each In some aspects, the agent is administered in a dosage amount of at least 1 mg/kg, 2 mg/kg, 4 mg/kg, 6 mg/kg, 8 mg/kg, 10 mg/kg or more.

In some embodiments, the agent(s) is/are administered by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

In some embodiments, the treatment further comprises bridging therapy, which is therapy between conditioning and the compositions disclosed herein or therapy administered after leukapheresis and completed prior to initiating conditioning chemotherapy. In some embodiments, the bridging therapy comprises, CHOP, G-CHOP, R-CHOP (rituximab, cyclophosphamide, doxorubicin, vincristine, and prednisolone), corticosteroids, bendamustine, platinum compounds, anthracyclines, and/or phosphoinositide 3-kinase (PI3K) inhibitors. In some embodiments, the PI3K inhibitor is selected from duvelisib, idelalisib, venetoclax, pictilisib (GDC-0941), copanlisib, PX-866, buparlisib (BKM120), pilaralisib (XL-147), GNE-317, Alpelisib (BYL719), INK1117, GSK2636771, AZD8186, SAR260301, and Taselisib (GDC-0032). In some embodiments, the AKT inhibitor is perifosine, MK-2206. In one embodiment, the mTOR inhibitor is selected from everolimus, sirolimus, temsirolimus, ridaforolimus. In some embodiments, the dual PI3K/mTOR inhibitor is selected from BEZ235, XL765, and GDC-0980. In some embodiments, the PI3K inhibitor is selected from duvelisib, idelalisib, venetoclax, pictilisib (GDC-0941), copanlisib, PX-866, buparlisib (BKM120), pilaralisib (XL-147), GNE-317, Alpelisib (BYL719), INK1117, GSK2636771, AZD8186, SAR260301, and Taselisib (GDC-0032).

In some embodiments, the bridging therapy comprises acalabrutinib, brentuximab vedotin, copanlisib hydrochloride, nelarabine, belinostat, bendamustine hydrochloride, carmustine, bleomycin sulfate, bortezomib, zanubrutinib, carmustine, chlorambucil, copanlisib hydrochloride, denileukin diftitox, dexamethasone, doxorubicin hydrochloride, duvelisib, pralatrexate, obinutuzumab, ibritumomab tiuxetan, ibrutinib, idelalisib, recombinant interferon alfa-2b, romidepsin, lenalidomide, mechloretamine hydrochloride, methotrexate, mogamulizumab-kpc, prerixafor, nelarabine, obinutuzumab, denileukin diftitox, pembrolizumab, plerixafor, polatuzumab vedotin-piiq, mogamulizumab-kpc, prednisone, rituximab, hyaluronidase, romidepsin, bortezomib, venetoclax, vinblastine sulfate, vorinostat, zanubrutinib, CHOP, COPP, CVP, EPOCH, R-EPOCH, HYPER-CVAD, ICE, R-ICE, R-CHOP, R-CVP, and combinations of the same.

In some embodiments, the cell immunotherapy is administered in conjunction with debulking therapy, which is used with the aim of reducing tumor burden. In one embodiment, debulking therapy is to be administered after leukapheresis and prior to administration of conditioning chemotherapy or cell infusion. Examples of debulking therapy include the following (Table 1):

TABLE 1
Exemplary debulking bridging therapies

| Type | Proposed Regimen^{a} | Timing/Washout |
|---|---|---|
| R-CHOP | Rituximab 375 mg/m² Day 1 | Should be administered |
| | Doxorubicin 50 mg/m² Day 1 | after leukapheresis/ |
| | Prednisone 100 mg Day 1 | enrollment and should |
| | through Day 5 | be completed at least |
| | Cyclophosphamide 750 mg/m² | 14 days prior to the |
| | Day 1 Vincristine 1.4 mg/m² | start of conditioning |
| | Day 1 | chemotherapy |
| R-ICE | Rituximab 375 mg/m² | |
| | Day 1 | |
| | Ifosfamide 5 g/m² 24 h-CI | |
| | Day 2 | |
| | Carboplatin AUC5 Day 2 | |
| | maximum dose 800 mg | |
| | Etoposide 100 mg/m²/d Days 1 | |
| | through Day 3 | |
| R-GEMOX | Rituximab 375 mg/m² Day 1 | |
| | Gemcitabine 1000 mg/m² Day 2 | |
| | Oxaliplatin 100 mg/m² Day 2 | |
| R-GDP | Rituximab 375 mg/m² | |
| | Day 1 (or Day 8) | |
| | Gemcitabine 1 g/m² on Day 1 | |
| | and Day 8 Dexamethasone 40 | |
| | mg on Day 1 through Day 4 | |
| | Cisplatin 75 mg/m² on Day 1 (or | |
| | carboplatin AUC5 on Day 1) | |
| RADIO- | Per local standard up to 20 to 30 | Should be administered |
| THERAPY^{b} | Gy | after leukapheresis/ |
| | | enrollment and should |
| | | be completed at |
| | | least 5 days prior to |
| | | the start of conditioning |
| | | chemotherapy |

Abbreviations: AUC, area under the curve
^{a}Other debulking treatment options may be used, to be discussed with the medical monitor. Supportive care with hydration, anti-emesis, mesna, growth factor support, and tumor lysis prophylaxis according to local standard may be used. More than 1 cycle allowed.
^{b}At least 1 target lesion should remain outside of the radiation field to allow for tumor measurements

### Monitoring

In some embodiments, administration of the immunotherapy (e.g., chimeric receptor T cell immunotherapy) occurs at a certified healthcare facility.

In some embodiments, the methods disclosed herein comprise monitoring patients at least daily for 7 days at the certified healthcare facility following infusion for signs and symptoms of CRS and neurologic toxicities and other adverse reactions to CAR T cell treatment. In some embodiments, the symptom of neurologic toxicity is selected from encephalopathy, headache, tremor, dizziness, aphasia, delirium, insomnia, and anxiety. In some embodiments, the symptom of adverse reaction is selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia. In some embodiments, patients are instructed to remain within proximity of the certified healthcare facility for at least 4 weeks following infusion.

### Prevention or Management of Adverse Events

In some embodiments, the method comprises management of adverse events in any subject. The terms “adverse events,” “adverse reaction,” and “adverse effect” are used interchangeably herein. In some embodiments, the adverse event is selected from the group consisting of cytokine release syndrome (CRS), a neurologic toxicity, a hypersensitivity reaction, a serious infection, a cytopenia and hypogammaglobulinemia. Methods for predicting, detecting, and/or managing adverse events associated with cell therapy treatment are known in the art and are described, by way of non-limiting example, in U.S. Pat. Nos. 6,905,874, 6,867,041, and 6,797,514, and in International Publication Nos. WO 2015/20096, WO 2016/191756, WO 2016/191755, WO 2019/079564, and WO 2021/092290, each of which are herein incorporated by reference in their entirety.

### Secondary Malignancies

In some embodiments, patients treated with CAR T cells (e.g., CD19-directed) or other genetically modified autologous T cell immunotherapy may develop secondary malignancies. In certain embodiments, patients treated with CAR T cells (.e.g, CD19-directed) or other genetically modified allogeneic T cell immunotherapy may develop secondary malignancies. In some embodiments, the method comprises monitoring life-long for secondary malignancies.

Methods and Compositions to Generate a Product for Increased Clinical Efficacy and/or Decreased Toxicity

In one embodiment, the disclosure provides a method of manufacturing an immunotherapy product with improved clinical efficacy and/or decreased toxicity to be used in patients according to the predicted grade of toxicity (CRS/NE).

By way of non-limiting example, methods for isolating immune cells and making CAR T cells for a therapeutic product from the isolated immune cells are disclosed in U.S. Patent Publication No. 2013/0287748, International Publication No. WO 2015/120096, International Publication No. WO 2016/191756, International Publication No. WO 2016/191755, International Publication No. WO 2019/079564, International Publication No. WO 2021/092290, and International Publication WO 2023/230276, each of which are herein incorporated by reference in their entirety.

In some embodiments, the immunotherapy product comprises blood cells. In some embodiments, blood cells collected from a subject or patient are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some embodiments, a washing step is accomplished a semi-automated “flow-through” centrifuge (for example, the Cobe 2991 cell processor, Baxter) according to the manufacturer's instructions. In some embodiments, a washing step is accomplished by tangential flow filtration (TFF) according to the manufacturer's instructions. In some embodiments, the cells are resuspended in a variety of biocompatible buffers after washing, such as, for example, Ca++Mg++free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient. In some embodiments, the methods include leukapheresis.

Optionally, in some embodiments, the method described herein further includes a step of enriching a population of lymphocytes obtained from the donor subject, prior to a transduction step. Enrichment of lymphocytes may be accomplished by any suitable separation method including, but not limited to, the use of a separation medium (e.g., Ficoll-Paque™, RosetteSep™ HLA Total Lymphocyte enrichment cocktail, Lymphocyte Separation Medium (LSA) (MP Biomedical Cat. No. 0850494X), a non-ionic iodixanol-based medium such as OptiPrep™, or the like), cell size, shape or density separation by filtration or elutriation, immunomagnetic separation (e.g., magnetic-activated cell sorting system, MACS), fluorescent separation (e.g., fluorescence activated cell sorting system, FACS), or bead-based column separation.

Optionally, in some embodiments, circulating lymphoma cells are removed from the sample through positive enrichment for CD4⁺/CD8⁺ cells via the use of selection reagents. In some such embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some such embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some embodiments includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps may be based on positive selection, in which the cells having bound the reagents are retained for further use, and/or negative selection, in which the cells having not bound to the antibody or binding partner are retained. In some examples, both fractions are retained for further use.

In some such embodiments, negative selection may be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step may deplete cells expressing multiple markers simultaneously, such as by incubating cells with a plurality of antibodies or binding partners, each specific for a marker targeted for negative selection. Likewise, multiple cell types may simultaneously be positively selected by incubating cells with a plurality of antibodies or binding partners expressed on the various cell types.

For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. For example, CD3+, CD28+ T cells may be positively selected using anti-CD3/anti-CD28 conjugated magnetic beads (e.g., DYNABEADS® M-450 CD3/CD28 T Cell Expander). In some embodiments, the population of cells is enriched for T cells with naïve phenotype (CD45RA+ CCR7+).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In some embodiments, T cells are separated from a PBMC sample by negative selection of markers expressed on non-T cells, such as B cells, monocytes, or other white blood cells, such as CD14. In some embodiments, a CD4+ or CD8+ selection step is used to separate CD4+ helper and CD8+ cytotoxic T cells. Such CD4+ and CD8+ populations may be further sorted into sub-populations by positive or negative selection for markers expressed or expressed to a relatively higher degree on one or more naive, memory, and/or effector T cell subpopulations.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques. In some embodiments, the sample or composition of cells to be separated is incubated with small, magnetizable or magnetically responsive material, such as magnetically responsive particles or microparticles, such as paramagnetic beads (e.g., such as Dynabeads™ or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample. In some embodiments, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some embodiments, a combination of positive and negative selection is performed during the same selection step, where the positive and negative fractions are retained and further processed or subject to further separation steps. In some embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies. In some embodiments, the magnetically responsive particles are left attached to the cells that are to be subsequently incubated, cultured and/or engineered; in some embodiments, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they may be eluted and recovered. In certain embodiments, the non-target cells are labelled and depleted from the heterogeneous population of cells.

In some embodiments, the isolation or separation is carried out using a system, device, or apparatus that carries out one or more of the isolation, cell preparation, separation, processing, incubation, culture, and/or formulation steps of the methods. In some embodiments, the system is used to carry out each of these steps in a closed or sterile environment, for example, to minimize error, user handling and/or contamination. In one example, the system is a system as described in International Patent Application, Publication Number WO2009/072003, or US 20110003380 Al, which are each incorporated herein by reference. In some embodiments, the system or apparatus carries out one or more, e.g., of the isolation, processing, engineering, and formulation steps in an integrated or self-contained system, and/or in an automated or programmable fashion. In some embodiments, the system or apparatus includes a computer and/or computer program in communication with the system or apparatus, which allows a user to program, control, assess the outcome of, and/or adjust various embodiments of the processing, isolation, engineering, and formulation steps. In some embodiments, the separation and/or other steps is carried out using CliniMACS system (Miltenyi Biotec), for example, for automated separation of cells on a clinical-scale level in a closed and sterile system. Components may include an integrated microcomputer, magnetic separation unit, peristaltic pump, and various pinch valves. The integrated computer in some embodiments controls components of the instrument and directs the system to perform repeated procedures in a standardized sequence. The magnetic separation unit in some embodiments includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some embodiments uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field and are collected within the cell collection bag.

In certain embodiments, separation and/or other steps are carried out using the CliniMACS Prodigy system (Miltenyi Biotec). The CliniMACS Prodigy system in some embodiments is equipped with a cell processing unity that permits automated washing and fractionation of cells by centrifugation. The CliniMACS Prodigy system may also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system may also include an integrated cell cultivation chamber which accomplishes cell culture protocols such as, e.g., cell differentiation and expansion, antigen loading, and long-term cell culture. Input ports may allow for the sterile removal and replenishment of media and cells may be monitored using an integrated microscope.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells may be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, at least 0.5×10⁹ lymphocytes are acquired from the donor and are optionally enriched and/or subjected to the stimulation. In some embodiments, at least 0.6×10⁹, 0.7×10⁹, 0.8×10⁹, 0.9×10⁹, 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2×10⁹, 2.5×10⁹, or 3×10⁹ lymphocytes are acquired from the donor, and are optionally enriched and/or subjected to the stimulation. In some embodiments, no more than 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2×10⁹, 2.5×10⁹, or 3×10⁹ lymphocytes are acquired from the donor and are optionally enriched and/or subjected to the stimulation.

In some embodiments, the incubation of the cells with any reagents for separating or isolating select cells is generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such a process and subsequent steps are carried out within an entirely closed system. In some such embodiments, a chamber is integral to the closed system. In some embodiments, this process (and in some embodiments also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

In some embodiments, the cells are incubated and/or cultured prior to or in connection with genetic engineering. The incubation steps may include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions may include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the cells are stimulated prior to or concurrently with a transduction step. In such embodiments, any combination of one or more suitable lymphocyte stimulating agents may be used to stimulate (activate) the lymphocytes. Non-limiting examples include an antibody or functional fragment thereof which targets a T-cell stimulatory or co-stimulatory molecule (e.g., anti-CD2 antibody, anti-CD3 antibody, anti-CD28 antibody, or functional fragments thereof) a T cell cytokine (e.g., any isolated, wildtype, or recombinant cytokines such as: interleukin 1 (IL-1), interleukin 2, (IL-2), interleukin 4 (IL-4), interleukin 5 (IL-5), interleukin 7 (IL-7), interleukin 15 (IL-15), tumor necrosis factor α (TNFα)), or any other suitable mitogen (e.g., tetradecanoyl phorbol acetate (TPA), phytohaemagglutinin (PHA), concanavalin A (conA), lipopolysaccharide (LPS), pokeweed mitogen (PWM)) or natural ligand to a T-cell stimulatory or co-stimulatory molecule. In some embodiments, the stimulating agent is an anti-CD3 antibody and/or an anti-CD28 antibody.

In some embodiments, the step of stimulating lymphocytes as described herein may entail stimulating the lymphocytes with one or more stimulating agents at a predetermined temperature, for a predetermined amount of time, and/or in the presence of a predetermined level of CO₂. In certain embodiments, the predetermined temperature for stimulation may be about 34° C., about 35° C., about 36° C., about 37° C., about 38° C., or about 39° C. In certain embodiments, the predetermined temperature for stimulation may be about 34-39° C. In certain embodiments, the step of stimulating the lymphocytes comprises stimulating the lymphocytes with one or more stimulating agents for a predetermined time. In certain embodiments, the predetermined time for stimulation may be about 24-72 hours. In certain embodiments, the predetermined time for stimulation may be about 24-36 hours. In certain embodiments, the step of stimulating the lymphocytes may comprise stimulating the lymphocytes with one or more stimulating agents in the presence of a predetermined level of CO₂. In certain embodiments, the predetermined level of CO₂ for stimulation may be about 1.0-10% CO₂. In certain embodiments, the predetermined level of CO₂ for stimulation may be about 1.0%, about 2.0%, about 3.0%, about 4.0%, about 5.0%, about 6.0%, about 7.0%, about 8.0%, about 9.0%, or about 10.0% CO₂.

In some embodiments, an anti-CD3 antibody (or functional fragment thereof), an anti-CD28 antibody (or functional fragment thereof), or a combination of anti-CD3 and anti-CD28 antibodies may be used in accordance with the step of stimulating the population of lymphocytes. Any soluble or immobilized anti-CD3 and/or anti-CD28 antibody or functional fragment thereof may be used (e.g., clone OKT3 (anti-CD3), clone 145-2C11 (anti-CD3), clone UCHT1 (anti-CD3), clone L293 (anti-CD28), clone 15E8 (anti-CD28)). In some aspects, the antibodies may be purchased commercially from vendors known in the art including, but not limited to, Miltenyi Biotec, BD Biosciences (e.g., MACS GMP CD3 pure 1 mg/mL, Part No. 170-076-116), and eBioscience, Inc. Further, one skilled in the art would understand how to produce an anti-CD3 and/or anti-CD28 antibody by standard methods. Any antibody used in the methods described herein should be produced under Good Manufacturing Practices (GMP) to conform to relevant agency guidelines for biologic products.

In certain embodiments, the T cell stimulating agent may include an anti-CD3 or anti-CD28 antibody at a concentration of from about 20 ng/mL-100 ng/mL. In certain embodiments, the concentration of anti-CD3 or anti-CD28 antibody may be about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL.

In some embodiments, the total duration of the incubation, e.g., with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours, or 72 hours. In some embodiments, the further incubation is for a time between or about between 1 hour and 48 hours, 4 hours and 36 hours, 8 hours and 30 hours, or 12 hours and 24 hours, inclusive.

In some embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the stimulation results in activation and/or proliferation of the cells, for example, prior to transduction.

In some embodiments, engineered cells, such as T cells, used in connection with the provided methods, uses, articles of manufacture or compositions are cells have been genetically engineered to express a recombinant receptor, e.g., a CAR or a TCR described herein. In some embodiments, the cells are engineered by introduction, delivery or transfer of nucleic acid sequences that encode the recombinant receptor and/or other molecules.

In some embodiments, methods for producing engineered cells includes the introduction of a polynucleotide encoding a recombinant receptor (e.g., anti-CD19 CAR) into a cell, e.g., such as a stimulated or activated cell. In particular embodiments, the recombinant proteins are recombinant receptors, such as any described. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems.

In some embodiments, the vectors can be viral vectors, such as lentiviral vectors, as well as retroviral vectors. Several recombinant viruses have been used as viral vectors to deliver genetic material to a cell. Viral vectors that may be used in accordance with the transduction step may be any ecotropic or amphotropic viral vector including, but not limited to, recombinant retroviral vectors, recombinant lentiviral vectors, recombinant adenoviral vectors, and recombinant adeno-associated viral (AAV) vectors. In one embodiment, the viral vector is an MSGV1 gamma retroviral vector. In some embodiments, the vectors are non-viral vectors.

In some embodiments, a total volume of at least 100 mL of the solution that contains the vector is used. In some embodiments, a total volume of at least 110 mL, 120 mL, 130 mL, 140 mL, 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 350 mL, or 400 mL of the solution that contains the vector is used. In some embodiments, a total volume of no more than 150 mL, 160 mL, 170 mL, 180 mL, 190 mL, 200 mL, 210 mL, 220 mL, 230 mL, 240 mL, 250 mL, 260 mL, 270 mL, 280 mL, 290 mL, 300 mL, 350 mL, 400 mL, or 500 mL of the solution that contains the vector is used.

In some embodiments, the vector solution includes at between 1×10³ to 1×10¹² transduction units per milliliter (TU/ml) of the viral vector.

In some embodiments, the lymphocyte transduction can be carried in the coated closed system with the immobilized vectors. In some embodiments, the transduction is performed with a sample that contained the lymphocytes. In some embodiments, the sample includes at least 2.5×10⁷ lymphocytes (e.g., T cells). In some embodiments, the sample includes at least 3×10⁷, 4×10⁷, 5×10⁷, 6×10⁷, 7×10⁷, 8×10⁷, 9×10⁷, 1×10⁸, 1.2×10⁸, 1.5×10⁸, 1.8×10⁸, 2×10⁸, 2.2×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.5×10⁸, 8×10⁸, 9×10⁸, or 10×10⁸ lymphocytes (e.g., T cells). In some embodiments, the sample includes no more than 3×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.5×10⁸, 8×10⁸, 9×10⁸, or 10×10⁸ lymphocytes (e.g., T cells).

In certain embodiments, the one or more compositions of stimulated T cells are or include two separate stimulated compositions of enriched T cells. In some embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In some embodiments, the two separate compositions include a composition of enriched CD8+ T cells. In some embodiments, two separate compositions of enriched CD4+ T cells and enriched CD8+ T cells are genetically engineered separately. In some embodiments, the same composition is enriched for both CD4+ T cells and CD8+ T cells and these are genetically engineered together.

In one embodiment, the sample of T lymphocytes is prepared by leukapheresis of PBMCs from the subject. In one embodiment, the leukapheresis sample is further subject to T lymphocyte enrichment through positive selection for CD4+ and/or CD8+ cells. In one embodiment, the lymphocytes are further engineered to comprise a CAR or an exogenous TCR. Examples of CARs and TCRs and methods of engineering lymphocytes are described elsewhere in the disclosure. In one embodiment, the method comprises expanding the engineered lymphocytes to produce a T cell infusion product in the presence of IL-2. In one embodiment, the engineered lymphocytes are expanded for about 1-7 days. In some embodiments, the expansion step includes IL-2.

Culturing of transduced lymphocytes can be done in media and conditions known in the art. In some embodiments, the culturing of the transduced lymphocytes may be performed at a temperature and/or in the presence of CO₂. In certain embodiments, the temperature may be about 34° C., about 35° C., about 36° C., about 37° C., about 38° C., or about 39° C. In certain embodiments, the temperature may be about 34-39° C. In certain embodiments, the predetermined temperature may be from about 35-37° C. In certain embodiments, the preferred predetermined temperature may be from about 36-38° C. In certain embodiments, the predetermined temperature may be about 36-37° C. or more preferably about 37° C.

In some embodiments, culturing of the transduced lymphocytes may be performed in the presence of a predetermined level of CO₂. In certain embodiments, the predetermined level of CO₂ may be 1.0-10% CO₂. In certain embodiments, the predetermined level of CO₂ may be about 1.0%, about 2.0%, about 3.0%, about 4.0%, about 5.0%, about 6.0%, about 7.0%, about 8.0%, about 9.0%, or about 10.0% CO₂. In certain embodiments, the predetermined level of CO₂ may be about 4.5-5.5% CO₂. In certain embodiments, the predetermined level of CO₂ may be about 5% CO₂. In certain embodiments, the predetermined level of CO₂ may be about 3.5%, about 4.0%, about 4.5%, about 5.0%, about 5.5%, or about 6.5% CO₂. In some embodiments, the step of expanding the population of transduced T cells may be performed at a predetermined temperature and/or in the presence of a predetermined level of CO₂ in any combination. For example, in one embodiment, the step of expanding the population of transduced T cells may comprise a predetermined temperature of about 36-38° C. and in the presence of a predetermined level of CO₂ of about 4.5-5.5% CO₂.

In some embodiments, the step of expanding the population of transduced T cells may range from at least one day, or at least 2 days, or at least 3 days, or at least 4 days, or at least 5 days, or at least 6 days, or at least 7 days. In some embodiments, the step of expanding the population of transduced T cells may be less than 1 day, or less than 2 days, or less than 3 days. In some embodiments, the step of expanding the population of transduced T cells occurs until the population of transduced cells has reached a predetermined number of viable cells, where the predetermined number is determined by a pre-determined therapeutic concentration or amount of cells needed for a therapy.

Any suitable culture medium T cell growth media may be used for culturing the cells in suspension. For example, a T cell growth media may include, but is not limited to, a sterile, low glucose solution that includes a suitable amount of buffer, magnesium, calcium, sodium pyruvate, and sodium bicarbonate. In one embodiment, the culturing media is OpTmizer™ (Life Technologies), but one skilled in the art would understand how to generate similar media.

In some embodiments, the stimulation is performed prior to the transduction step. In some embodiments, the stimulation is performed after the transduction step.

In some embodiments, the separation, incubation and/or transduction steps can be carried out in a closed system, without limitation. In certain embodiments, the closed system is a closed bag culture system, using any suitable cell culture bags (e.g., Mitenyi Biotec MACS® GMP Cell Differentiation Bags, Origen Biomedical PermaLife™ Cell Culture bags).

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some embodiments may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts, genomic database sequences, and scientific literature cited herein are hereby incorporated by reference. All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control.

The following examples are intended to illustrate various aspects of the application. As such, the specific aspects discussed are not to be construed as limitations on the scope of the application. For example, although the Examples below are directed to T cells transduced with an anti-CD19 chimeric antigen receptor (CAR), one skilled in the art would understand that the methods described herein may apply to immune cells transduced with any CAR. The methods are also applicable to other immunotherapies. It will be apparent to one skilled in the art that various equivalents, changes, and modifications may be made without departing from the scope of application, and it is understood that such equivalent aspects are to be included herein.

The disclosures provided by this application may be used in a variety of methods in additional to, or as a combination of, the methods described above. The following is a compilation of exemplary methods that may be derived from the disclosures provided in this application.

### EXAMPLES

### Example 1

Autologous anti-CD19 CAR T cell therapy is a curative-intent treatment for B cell malignancies. Still, over 50% of patients either fail to respond or relapse. Tumor intrinsic factors like high disease burden, low antigen expression, or an immune-suppressive microenvironment are associated with disease progression. A less differentiated, naïve-like T cell phenotype and CAR T cell expansion are linked to favorable outcome. These factors, however, could only partially explain treatment outcome, and understanding of product features associated with resistance remains limited.

Here, we performed genomic and functional assessments of axicabtagene ciloleucel (axi-cel) products from 36 LBCL patients in ZUMA-1. Genomic evaluation included both single-cell RNA sequencing (scRNAseq) and epigenetic sequencing (scATACseq). Functional assays measured cytokine secretion after CAR T cell stimulation with CD19-coated red blood cells (RBC) or CD19+ cancer cells. Additionally, tumor bearing NSG-MHC I/II DKO mice were treated with axi-cel from 19 patients to evaluate in vivo efficacy, including CAR T-cell pharmacokinetics and cytokine levels. Correlative analyses between assay results and matched patients' clinical outcomes were performed.

We found that CD19 antigen stimulation in vitro induced production of Th1 (IL2, IFNγ) and Th2 (IL4, IL5, IL13) cytokines, highlighting differences in Th1 polarization. A scaled Th1-Th2 index was strongly linked to clinical efficacy (P<0.05), especially with CD19-cross-linked RBC stimulation. scRNA-seq of axi-cel products from ZUMA-1 patients revealed 12 transcriptionally distinct T cell clusters. We identified strong association (P=0.029) between disease progression and a CD8+ cluster overexpressing Th2 master regulator, GATA3. Additionally, a hypoproliferative exhausted T cell cluster was found to be associated with lack of response. Both clusters showed enrichment of exhaustion marker, TIGIT. Matched scATAC-seq confirmed these findings and presented an enrichment in chromatin accessibility in the GATA3 promoter of CD8+ T cells from non-responders. Lastly, tumor-bearing mice treated with CAR T products from patients with durable responses showed superior tumor clearance and survival, compared to those treated with products from relapsed or non-responder patients. Patient clinical response was associated with mouse survival (P=0.08). Further, tumor control in vivo significantly correlated with CAR T cell expansion in both mice and patients (P=0.01), as well as with the Th1-Th2 index in mice and patients.

These functional assays, as well as transcriptomic and epigenetic profiling with axi-cel, demonstrate the importance and relative ranking of product features in driving treatment outcomes. These findings can inform on manufacturing processes for developing more efficacious CAR-T cell products, and optimization of treatment protocols to better serve patients at risk of non-response or relapse following CAR-T cell therapy.

### Results

Subset of Product Samples Analyzed in this Study are Representative of ZUMA1 Clinical Response

Tumor-intrinsic factors, including elevated disease burden, low antigen expression, and an immunosuppressive microenvironment, have been linked to disease progression. Additionally, a less differentiated, naïve-like T cell phenotype, along with robust CAR-T cell expansion, has been associated with improved outcomes. However, these characteristics only partially explain the outcomes following CAR-T cell therapy, and the understanding of product features that may predict or contribute to treatment resistance remains limited.

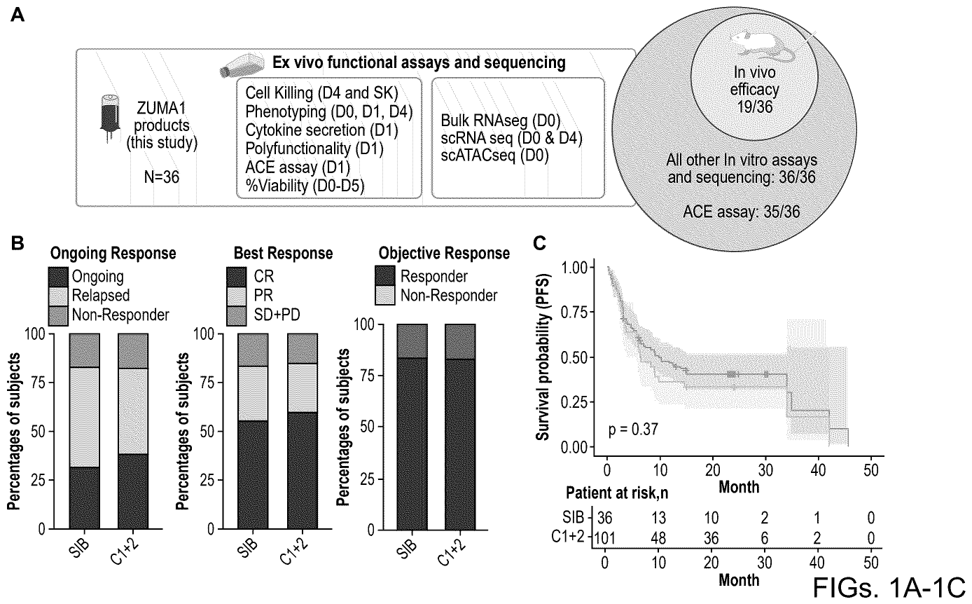

Secondary infusion bags from ZUMA1 patients present a unique opportunity to investigate Axi-cel products in greater depth, including through genomic and functional assessments, both in vitro and in vivo (FIG. 1A).

In this study, Axi-cel products from 36 patients were evaluated. The clinical responses in this subset—measured by ongoing response, best response, and objective response (FIG. 1B)—showed a distribution comparable to those observed in the ZUMA1 pivotal cohorts (cohorts 1 and 2). Additionally, there was no significant difference in progression-free survival (PFS) between these 36 patients and those from the original ZUMA1 study (FIG. 1C).

FIGS. 1A-1C are schematics and graphs showing that a subset of product samples analyzed represents the clinical response in ZUMA1. FIG. 1A is a schematic of ex vivo functional analyses of ZUMA1 SIB products. FIG. 1B shows a comparison of clinical responses between ZUMA1 C1+2 patients and the subset with SIB products. FIG. 1C shows the progression-free survival (PFS) of ZUMA1 C1+2 patients compared to those with SIB products.

### CD19 Antigen Stimulation of CAR T Products Highlighted Differences in Th1 and Th2 Polarization

We tested products from 36 subjects using a range of assays. Many, such as cell-killing assays, showed no correlation with clinical response (not shown). However, product phenotyping revealed a trend where ongoing responders had a higher frequency of naïve CAR-T cells, supporting previous findings (not shown).

Among the assays that demonstrated a significant association with clinical outcomes, we found differences in Th1 and Th2 cytokine secretion between ongoing responders and those who relapsed or did not respond. Cytokine levels were measured from the supernatant after co-culturing the products with target cells. Non-responders exhibited trends of higher secretion of Th2 cytokines (IL-4, IL-5, IL-13) compared to ongoing responders and those who relapsed (not shown).

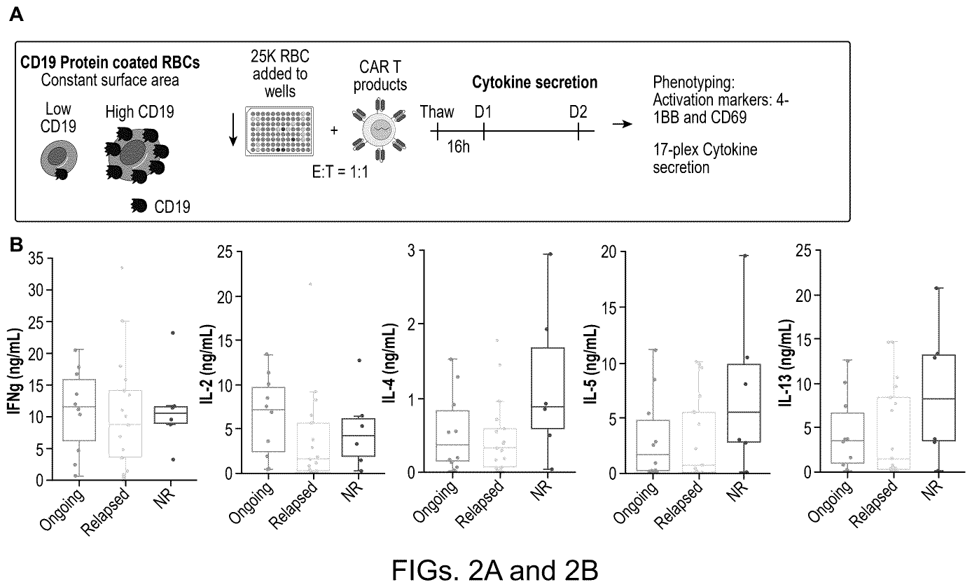

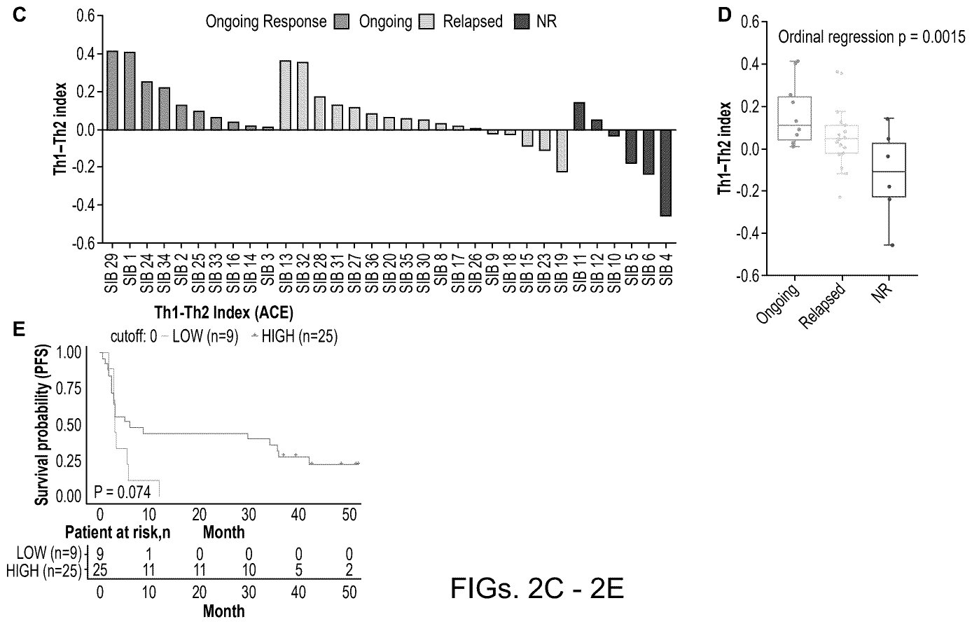

To eliminate confounding factors from co-stimulation and soluble elements, we used a co-culture assay with CD19 (antigen)-coated erythrocytes to measure cytokines, referred to as ACE assay from hereon (FIG. 2A). We also performed phenotyping of activation markers, CD69 and 4-1BB, by flow cytometry after overnight co-culture with CD19-coated erythrocytes. Distinct differences emerged in the secretion of Th1 and Th2 cytokines: ongoing responders tended to have higher levels of Th1 cytokines (IL-2, IFNg), while relapsed and non-responders showed increased Th2 cytokines (IL-4, IL-5, IL-13) (FIG. 2B). However, no differences were observed in CD69 or 4-1BB expression across the clinical response groups (not shown).

We developed a Th1-Th2 index to further explore the correlation between these cytokines and clinical response. The index was calculated as follows:

$$Th 1 - Th 2 = ( IFNg + IL - 2 ) / 2 - ( IL - 4 + IL - 5 + IL - 13 ) / 3$$

At the subject level, all ongoing responders had a positive index, while relapsed and non-responders exhibited a mix of positive and negative indices (FIG. 2C). The Th1-Th2 index was significantly higher in ongoing responders compared to relapsed and non-responders (FIG. 2D). Moreover, subjects with a positive Th1-Th2 index had significantly better progression-free survival (PFS) compared to those with a negative index (FIG. 2E).

FIGS. 2A-2E are a schematic and graphs showing that in vitro measurement of Th1 and Th2 cytokines correlates with clinical response. FIG. 2A is a schematic of antigen (CD19)-coated erythrocytes assay. FIG. 2B shows levels of IFN-γ, IL-2, IL-4, IL-5, and IL-13 cytokines in the ACE assay by ongoing response (n=34). In FIG. 2C, Th1-Th2 index was calculated using Th1 and Th2 cytokine levels for all subjects (n=34; ordered from high to low within each response group). In FIG. 2D, the Th1-Th2 index is based on ongoing response (n=34). FIG. 2E shows Kaplan-Meier estimated progression-free survival (PFS) for positive vs. negative Th1-Th2 index.

Additionally, the Th1-Th2 index calculated from serum cytokine measurements in patients showed a similar trend, being significantly higher in subjects with ongoing response and improved survival (not shown). There was also a strong correlation between the Th1-Th2 index measured by the ACE assay in the co-culture system and the serum cytokines in ZUMA1 patients.

scRNAseq of Products Identify Dysfunctional Immune Regulation Associated with Disease Progression

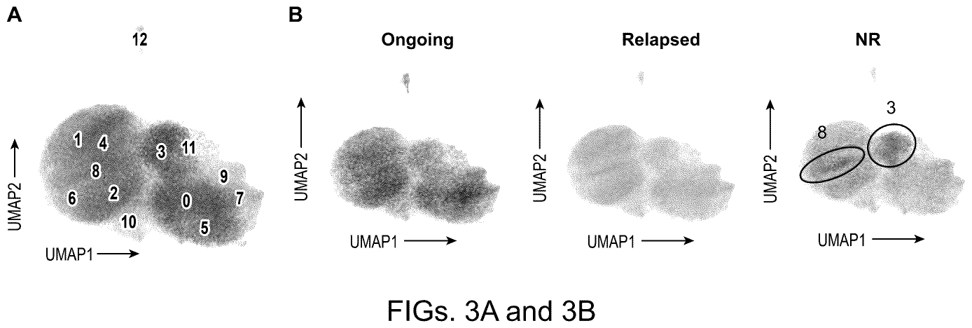

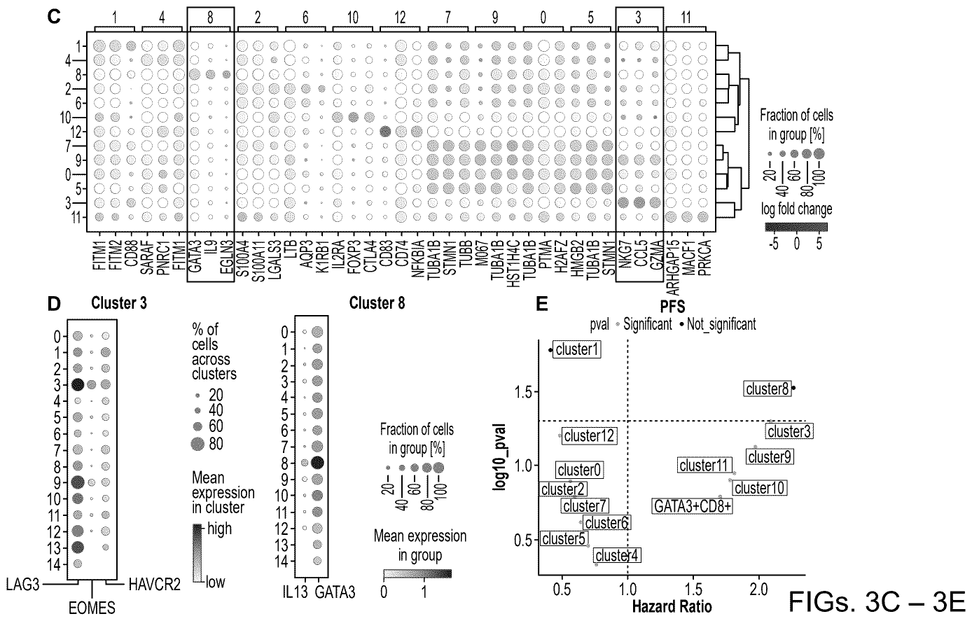

Baseline samples from 36 SIBs were processed and sequenced for scRNAseq. Downstream analysis was performed with Scanpy (scanpy.readthedocs.io/en/stable/), following standard procedures such as doublet filtering based on gene counts and batch correction using Harmony (github.com/immunogenomics/harmony). After quality control and filtering, 189,106 cells were obtained across the 36 samples, with an average of 5,252 cells per sample. The data was normalized, log-transformed, and processed for feature selection, PCA, and UMAP visualization. No clustering bias was observed by patient or patient CAR-T peak of expansion (not shown). Leiden clustering identified 13 major cell groups (FIG. 3A).

To explore potential patterns related to clinical outcomes, we mapped ongoing response patterns on UMAP (FIG. 3B) and observed an enrichment of non-responders in scRNA-seq clusters 3 and 8 (not shown). Differentially expressed genes were identified for each cluster using the “get markers” function, generating a ranked list of the most highly expressed genes. The top three markers revealed cell identities associated with proliferation, effector T-cells, and conventional T-cell markers (FIG. 3C). To refine cell identification, we employed the CellTypist label transfer method (celltypist.org), using data from Deng et al., 2021, which provided more comprehensive cell labeling (not shown).

Cluster 3 exhibits high expression of effector markers (FIG. 3C) along with strongest expression and detection of exhaustion markers (FIG. 3D). While cluster 9 also show effector genes as top markers, the downregulation of proliferative genes in cluster 3 suggests that cells in this cluster from non-responders are linked to reduced renewal capacity (FIG. 3C). Non-responder cells were also enriched in cluster 8 which has an increased expression of GATA3, IL9R and EGLN3. GATA3 expression is usually associated with Th2 subtypes, but expression patterns of CD8 (FIG. 3C) showed that, GATA3 CD8 cells were a major component of cluster 8. While GATA3 is expressed across all clusters, cluster 8 displays the highest expression levels (FIG. 3D).

In addition to analyzing ongoing responses, we examined the association of all clusters with PFS. By categorizing each cluster based on its percentage, ensuring at least 40% of subjects in each group, we observed that clusters 3 and 8 had a positive hazard ratio (HR), whereas cluster 1 exhibited a negative HR (not shown). This suggests that clusters 3 and 8 are linked to disease progression, while cluster 1 is associated with improved survival outcomes (FIG. 3E), aligning with its association with ongoing response. Cluster 1, representing the Tscm phenotype (not shown), has been previously associated with favorable clinical responses.

FIGS. 3A-3E are schematics and graphs showing the scRNAseq of products identify dysfunctional immune regulation associated with disease progression. FIG. 3A is a UMAP of scRNAseq leiden clusters. FIG. 3B is a UMAP of clusters by ongoing response. FIG. 3C show the top 3 genes by each cluster. FIG. 3D shows additional genes enriched in clusters 3 and 8. FIG. 3E is a volcano plot showing association of clusters with KM-estimated PFS (minimum proportion is 40% per group, categorical variable).

Clusters Identified Through scATAC-Seq Corroborate the Findings on Dysfunctional GATA3+ CD8+ T-Cells

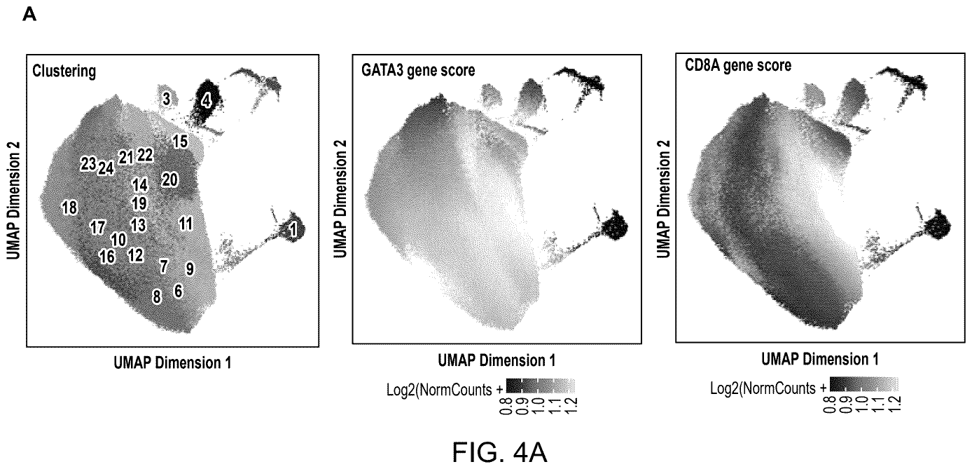

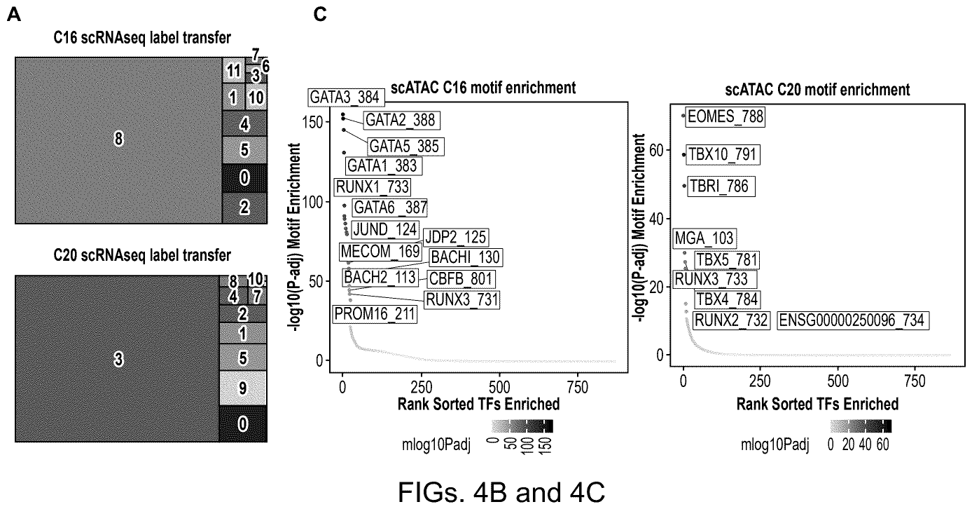

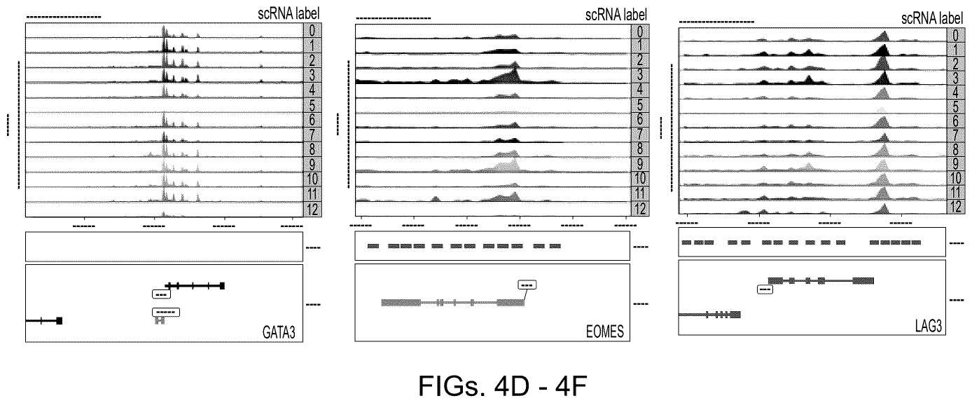

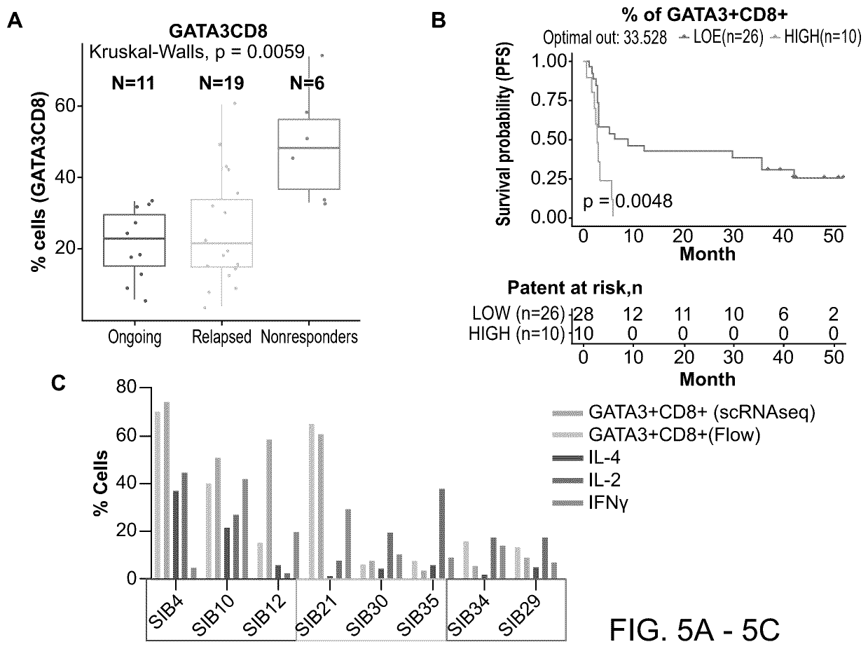

In addition to scRNA-seq, we performed a single-cell assay for transposase-accessible chromatin (scATAC-seq) on axi-cel products from matched patients. This analysis identified 24 distinct T-cell clusters across 231,967 cells (FIG. 4A). Using ArchR to calculate gene scores based on chromatin accessibility patterns, we found that cluster 16 had higher GATA3 and CD8 scores compared to other clusters (FIG. 4A), supporting the scRNA-seq findings. Although GATA3 expression was observed across most cells, increased chromatin accessibility at the GATA3 locus was noted specifically in scRNA-seq cluster 8 and scATAC-seq cluster 16. The initial classification of scATAC-seq clusters was based on chromatin accessibility at the CD4 and/or CD8A loci (not shown) and motif enrichment analysis for each cluster (not shown).

Further motif analysis for each cluster, verified that C16 was enriched in GATA family motifs, GATA3 being the top hit. The gene body of GATA3 was open across the different cells identified with their correspondent scRNA-seq group, difference in expression and gene score could originate by an increase in chromatin opening in the promotor region of GATA3 (FIG. 4D).

In a similar fashion, C20 motif enrichment supported EOMES as the top hit, implying that cells in this cluster are the label assigned by scRNA-seq. EOMES gene body is particularly open at cells labeled as cluster 3, 9 and 11 from scRNA-seq (FIG. 4E, F), but other exhaustion genes didn't show specific patterns to cells labeled as exhausted (not shown).

FIGS. 4A-4F are schematics and graphs showing that corresponding clusters of scATACseq validate findings on GATA3+CD8+ dysfunctional T-cells. FIG. 4A is a scATAC-seq UMAP colored by (left to right) cluster, GATA3 gene score and CD8A gene score. FIG. 4B shows corresponding scRNAseq clusters in scATACseq for cluster C16 (top) and C20 (bottom). FIG. 4C shows motif enrichment of C16 and C20. FIG. 4D shows chromatin track for GATA3 upstream region across scATAC cells labeled by scRNA-seq. FIG. 4E shows EOMES gene body chromatin track across scATAC cells labeled by scRNA-seq. FIG. 4F shows LAG3 gene body chromatin track across scATAC cells labeled by scRNA-seq.

### Comparison of GATA3 Expression at RNA and Protein Level

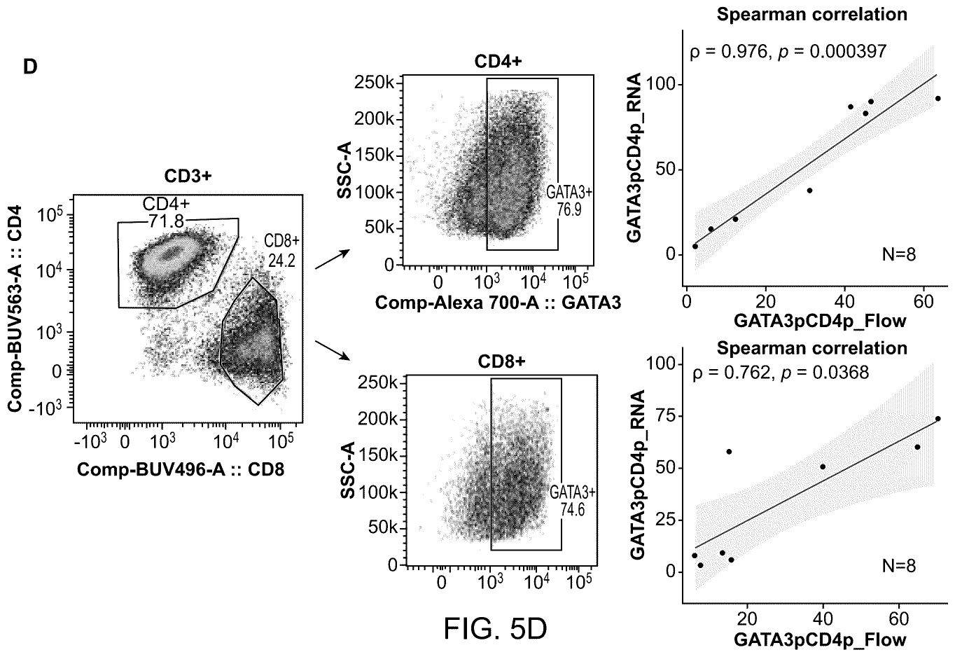

The GATA3+CD8+ T cell subset identified in the scRNAseq and scATACseq was shown to be enriched in non-responders compared to ongoing and relapsed subjects (FIG. 5A). When using an optimal cutoff for frequency of GATA3+CD8+ T cells by scRNAseq data, it was shown that subjects with high GATA3+CD8+ frequency had significantly shorter PFS compared to subjects with low frequency of GATA3+CD8+ cells (FIG. 5B). To confirm scRNAseq and scATACseq GATA3+CD8+ results, flow cytometry assessment of T cell subsets before and after PMA+Ionomycin was performed. Flow cytometry analysis confirmed the results seen in scRNAseq data, and subjects for which high GATA3+CD8+ frequency was observed by sequencing also had high frequency by flow. T cell functionality was also assessed by flow cytometry to determine if high level Th2 cytokine secretion seen in the CD19 ACE assay data could also be seen due to PMA+Ionomycin stimulation. With flow cytometry we were able to discern if CD4s or CD8s were producing the Th2 cytokines as well. In the case of non-responders with high Th2 cytokine production, like SIB4 & SIB10, the flow results recapitulated other functional assessments with high levels of the Th2 cytokine, IL-4, being produced by these subjects. While for other non-responders with less Th2 cytokine skew, SIB12, not having robust production of IL-4 post stimulation (FIG. 5C). Interestingly, in our functional assessment the distinguishing factor of non-responders SIB4 and SIB10 was that CD8 T cells were making high levels of Th2 cytokine, IL-4, while subjects across response groups had varying levels of IL-4 production by CD4s. To determine the robustness of the flow cytometry-based measurements of GATA3 compared to scRNAseq, GATA3 flow data was compared to scRNAseq, and both CD4 and CD8 GATA3 expression showed strong concordance between flow and scRNAseq methods (FIG. 5D).

FIGS. 5A-5D are graphs showing quantification of dysfunctional GATA3+CD8+ T cells through immunophenotyping. FIG. 5A shows frequencies of GATA3+CD8+ cells assessed using scRNAseq by ongoing response. FIG. 5B shows Kaplan-Meier (KM) analysis of progression-free survival (PFS) based on the percentage of GATA3+CD8+ cells determined by scRNAseq at an optimal cutoff. FIG. 5C is a comparison of GATA3+CD8+ cell frequencies measured by scRNAseq, flow cytometry, and functional cytokines IL4, IL2, and IFN-γ in a subset of participants. FIG. 5D shows correlation analysis of GATA3+CD8+ and GATA3+CD4+ populations using scRNAseq and flow cytometry.

### Axi-Cel Administered to ZUMA1 Patients Showed Similar Efficacy in a Disseminated Raji B-Cell Lymphoma In Vivo Model

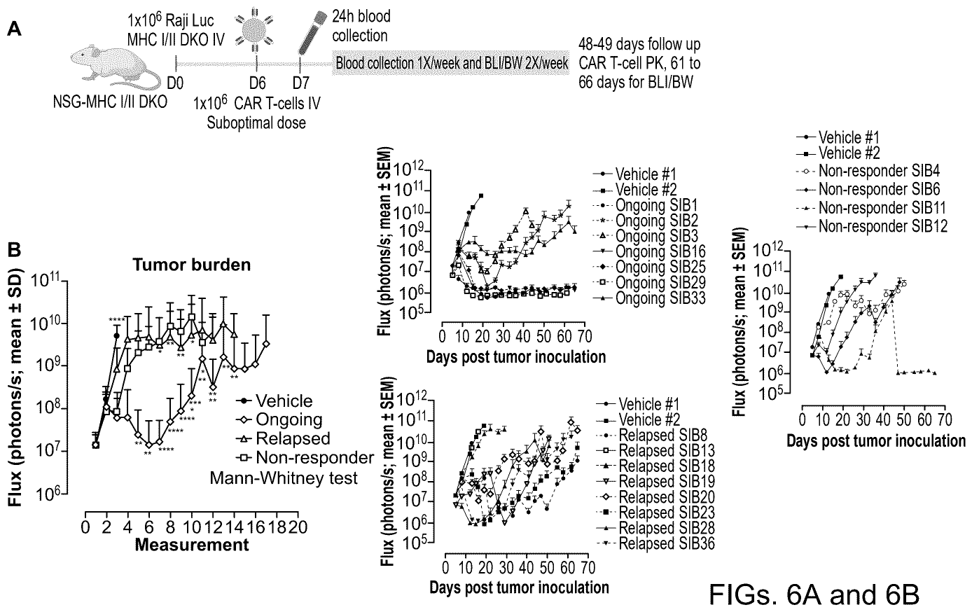

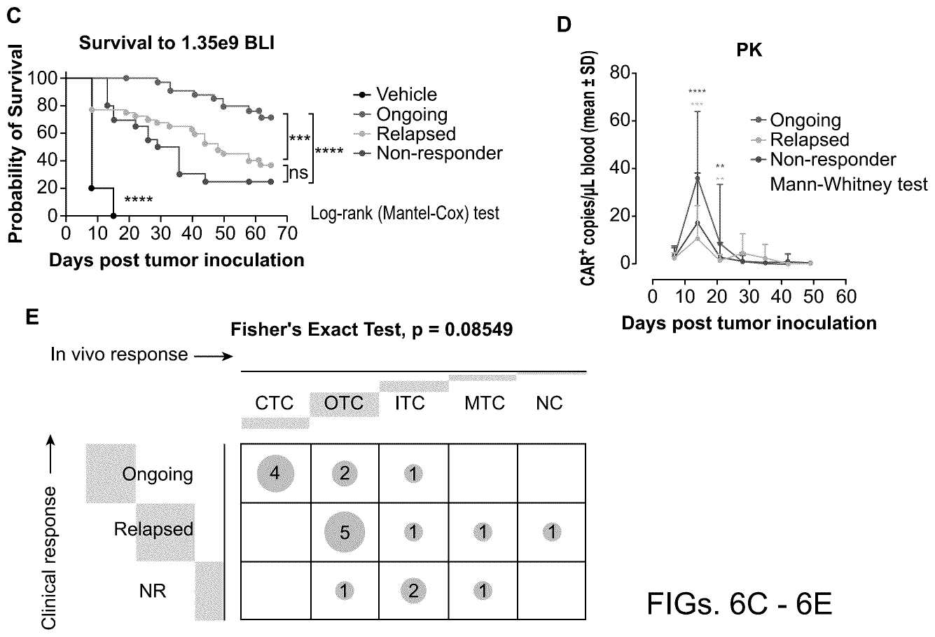

We evaluated the in vivo anti-tumor response of 19 ZUMA-1 SIB products (7 ongoing, 8 relapsed and 4 non-responders) to determine if a correlation with the clinical response would exist. NSG-MHC I/II DKO mice were inoculated intravenously with Raji Luc MHC I/II DKO tumor cells to establish a disseminated B-cell lymphoma model. Treatment consisted of intravenous injection of either vehicle (PBS) or a sub-efficacious dose of one million CAR T cells and was initiated on day 6 post tumor inoculation when all mice showed evidence of systemic disease, as indicated by whole body bioluminescence imaging (FIG. 6A). Along with 19 SIB products, 3 CAR T products manufactured from healthy donor cells were also evaluated (data not shown). Tumor burden was quantified using bioluminescence imaging twice weekly and blood samples were collected 24 h post treatment and weekly thereafter to quantify CAR T cells by ddPCR. Body weight was measured twice weekly and expressed as change from initial body weight over time (FIG. 6A).

Mice in the vehicle control groups succumbed to disease within 2 to 3 weeks post tumor implantation (3^{rd} measurement) (FIG. 6B). In comparison, all mice that received axi-cel products had significantly lower disease burden by the 3^{rd} measurement (FIG. 6B). Treatment with axi-cel products from ongoing responders achieved complete tumor control for 4 out of 7 SIB products tested and showed overall statistically superior efficacy and survival compared to products from non-responders and relapsed subjects. Products from relapsed and non-responders led to delayed tumor growth or transient tumor control followed by relapse (at the exception of complete control by study end for 1 out of 4 SIB non-responder products, and none for relapsed subjects) (FIGS. 6B and 6C). The anti-tumor responses in the mice were heterogeneous, both at individual mouse level and at subject level (FIG. 6B, FIG. 1C). Axi-cel infusion in mice was well-tolerated, as indicated by maintenance of body weight over time (not shown) without any detectable clinical sign related to other than high tumor burden.

All mice treated with axi-cel observed a CAR copy peak expansion in the peripheral blood 1 week following infusion, followed by decline over time. The ongoing responder group showed statistically higher CAR peak expansion compared to relapsed and non-responder groups, in correlation with the observed anti-tumor activity and survival (FIG. 6D). In vivo anti-tumor responses showed correlation with clinical response by Fisher exact test (p=0.085) with only 19 subjects (FIG. 6E).

FIGS. 6A-6E are schematics and graphs showing that Axi-cel products administered to ZUMA1 patients showed similar efficacy in a systemic human B-cell lymphoma in vivo model. FIG. 6A is a schematic of the systemic Raji B-cell lymphoma model in NSG-MHC I/II DKO mice, axi-cel treatment, and analyses. In FIG. 6B, tumor burden was monitored with bioluminescence imaging and recorded as photons/second, at each incremental measurement twice a week, each corresponding to 1 or 2 consecutive days post tumor inoculation for each group: left panel: means of pooled groups of n=5 tumor-bearing mice treated with vehicle (2 groups) or axi-cel products from 7 ongoing responders, 8 relapsed subjects, or 4 non-responding patients, and right panel: means of individual groups of n=5 mice. Statistical analysis performed with the Mann-Whitney test. FIG. 6C shows Kaplan-Meier survival analysis set at a tumor burden <1.35×10⁹ photons/second bioluminescence. Statistical analysis performed with the Mantel-Cox log-rank test. FIG. 6D shows mouse peripheral blood pharmacokinetic analysis of CAR T cells over time as mean CAR copies/μL blood analyzed by ddPCR. Statistical analysis performed with the Mann-Whitney test. FIG. 6E is a contingency table showing association of clinical response in patients with anti-tumor efficacy in vivo. Data are combined from 2 in vivo studies. BLI: bioluminescence imaging, BW: body weight, CTC: complete tumor control, ITC: intermediate tumor control response (tumor burden got controlled followed by relapse in 5/5 mice), MTC: minimal tumor control (tumor growth delay), NC: no tumor control (similar to vehicle group), NR: non-responder, OTC: overall tumor control (not 5/5 mice showed complete tumor control but at least 2/5 achieved a complete response), PK: pharmacokinetic. ns P>0.05, * P<0.05, ** P<0.01, *** P<0.001, **** P<0.0001.

Th1-Th2 Index Measured In Vivo Associate with Clinical Response

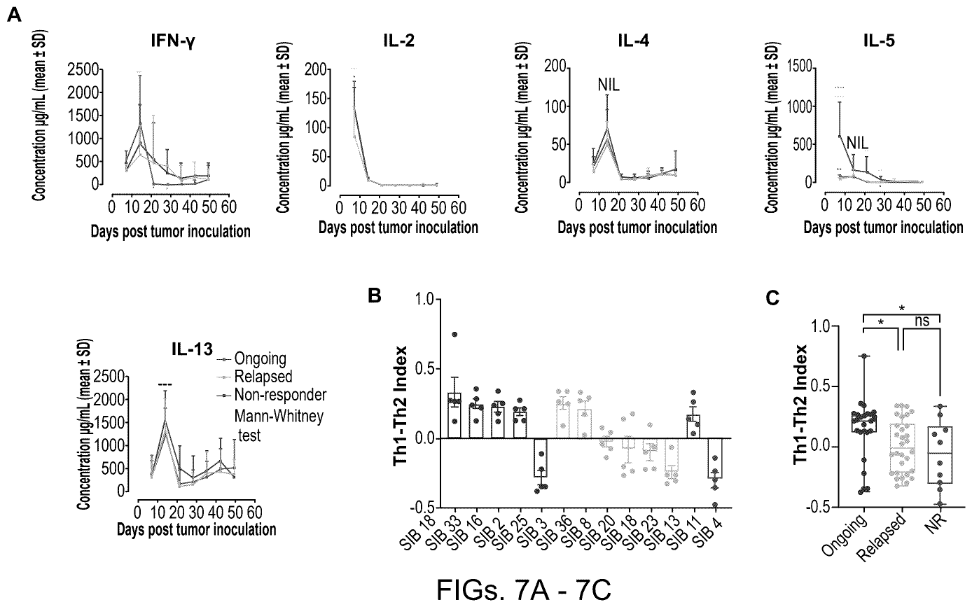

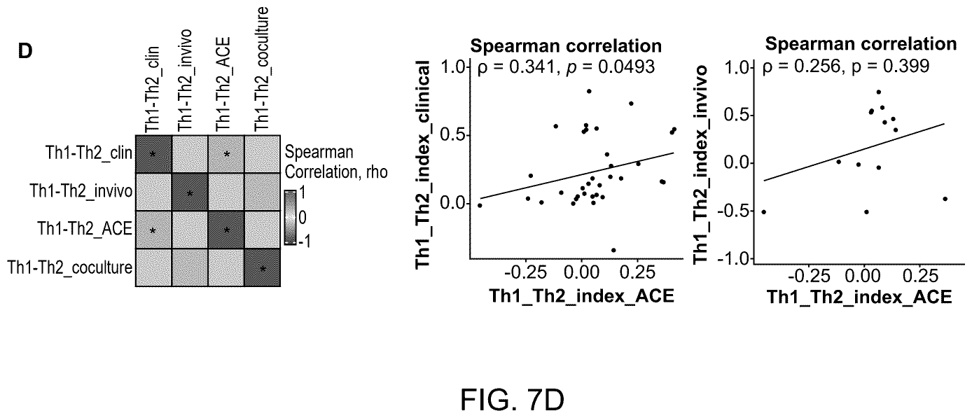

Blood samples were collected from all mice at various time points to measure Th1 and Th2 cytokines in the plasma. Ongoing responders exhibited statistically elevated levels of Th1 cytokines, particularly IL2 and IFNg, while non-responders had higher levels of the Th2 cytokine IL-5, in agreement with ACE assay findings (FIG. 7A).

The Th1-Th2 index, calculated from in vivo cytokine measurements (n=13 SIB products), revealed a predominantly positive index in ongoing responders, except for SIB3. In contrast, relapsed and non-responders showed a mix of positive and negative indices (FIG. 7B). The index was significantly higher in ongoing responders compared to both relapsed and non-responders (FIG. 7C).

We further analyzed the correlation of Th1 and Th2 cytokines, along with the Th1-Th2 index, across multiple platforms including the ACE assay, co-culture experiments, and measurements in mice and patients. Strong correlations were observed between the different platforms (FIG. 7D).

FIGS. 7A-7D are graphs showing that the Th1-Th2 index measured in vivo associate with clinical response. FIG. 7A shows mean plasma levels of IFN-γ, IL-2, IL-4, IL-5, and IL-13 in tumor-bearing mice treated with axi-cel products from 5 ongoing responders, 6 relapsed, or 2 non-responding subjects, at various timepoints following tumor inoculation. FIG. 7B shows the Th1-Th2 index derived from in vivo cytokine secretion in axi-cel-treated mice (n=13 SIB products tested; ordered from high to low in each response group). FIG. 7C shows the Th1-Th2 index measured in vivo at individual mouse level for ongoing responders vs relapsed and non-responders. FIG. 7D shows the Spearman correlation of Th1-Th2 index across multiple platforms. In FIGS. 7A-7D, Th1-Th2 index in vivo was determined by measuring peak cytokine secretion from one tumor-bearing mouse (n=5 per group) treated with the axi-cel product from 6 ongoing responders, 5 relapsed, or 2 non-responding subjects and normalized to a value between 0-1. Index was calculated as follows: Th1-Th2=(IFN-γ+IL-2)/2−(IL-4+IL-5+IL-13)/3.

Association and Ranking of Product Features Associated with Clinical Response

We analyzed product features across 36 products to investigate the associations between covariates and rank parameters based on ongoing response and progression-free survival (PFS). Ordinal regression revealed that the Th1-Th2 index measured in patients was the strongest predictor of ongoing response, followed by CAR T peak expansion in patients, the Th1-Th2 index in the ACE assay, Th1 cytokines (IL2 and IFNg levels) in patients, in vivo efficacy, and CAR T expansion in mice. Conversely, the percentage of GATA3+CD8+ T cells was the top covariate associated with disease progression, followed by Th2 cytokine IL5 measured in serum of CAR T cells co-cultured with CD19+ cancer cells, as well as scRNAseq clusters 3 and 8. These parameters showed similar associations when ranked by PFS using Cox regression and log-rank tests.

Beyond ranking features by their association with response, we also explored correlations between them. In vivo efficacy correlated significantly with the percentage of naïve CAR T cells, CAR T peak expansion in both mice and patients, and the Th1-Th2 index measured in both the ACE assay and in mice. The Th1-Th2 index across different platforms was also correlated with CAR T peak expansion and the percentage of naïve CAR T cells in the product. In contrast, GATA3+CD8+ T cells were negatively correlated with the Th1-Th2 index in the ACE assay. Covariates associated with clinical response ranked higher than the percentage of naïve T cells, although a trend of positive association (not statistically significant) was noted for naïve T cells. Moreover, there was no correlation between GATA3+CD8+ cells and naïve CAR T cells, suggesting that GATA3+CD8+ cells are an independent prognostic factor associated with response.

### Example 2

The results from the example described above are also used to improve or optimize CAR-T cell treatment by informing the CAR-T cell manufacturing process.

For example, an example of such an optimization embodiment includes a method for manufacturing transformed lymphocytes, including the steps of (1) measuring an expression level of GATA3 in a sample of lymphocytes collected from a subject, (2) depleting cells expressing CD45RO from the sample of lymphocytes if the expression level of GATA3 in the sample of lymphocytes is above a control value, and (3) incubating the sample of lymphocytes with a polynucleotide vector to transform the sample of lymphocytes to produce transformed lymphocytes. In such an embodiment, the step of depleting cells expressing CD45RO from the sample of lymphocytes is carried out if the percentage of GATA3+CD8+ cells in the sample of lymphocytes is at least 50.0% of the total lymphocyte population in the sample. Alternatively, in such an embodiment, the step of depleting cells expressing CD45RO from the sample of lymphocytes is carried out if the percentage of GATA3+CD8+ cells in the sample of lymphocytes is at least 33.5% of the total lymphocyte population in the sample. Alternatively, in such an embodiment, the step of depleting cells expressing CD45RO from the sample of lymphocytes is carried out if the percentage of GATA3+CD8+ cells in the sample of lymphocytes is at least 20.0% of the total lymphocyte population in the sample. Alternatively, in such an embodiment, the step of depleting cells expressing CD45RO from the sample of lymphocytes is carried out if the percentage of GATA3+CD8+ cells in the sample of lymphocytes is at least 10.0% of the total lymphocyte population in the sample.

In such an embodiment, the step of depleting cells expressing CD45RO from the sample of lymphocytes is completed before the sample of lymphocytes is incubated with the polynucleotide vector. Also, the step of depleting cells expressing CD45RO from the sample of lymphocytes includes the steps of contacting the sample of lymphocytes with an anti-hCD45RO antibody and bead conjugate to generate a bead-bound population of cells expressing CD45RO, and separating the bead-bound population of cells expressing CD45RO from the sample of lymphocytes.

In such an embodiment, the polynucleotide vector is a retroviral or lentiviral vector and encodes an anti-CD19 chimeric antigen receptor (CAR).

In such an embodiment, an activation step is additionally included if a retrovirus is used to transduce the polynucleotide vector into the lymphocytes. The activation step is accomplished by stimulating the sample of lymphocytes with at least one T cell stimulating agent such as an anti-CD3 antibody, an anti-CD28 antibody, or Interleukin-2, or combinations thereof.

The transformed or transduced lymphocytes can be cultured for a maximum of 72 hours, 48 hours, or 36 hours if there is a need to generate a final immunotherapy product from the transformed or transduced lymphocytes in an expedited manner.

Another example of such optimization embodiment includes a method for manufacturing transformed lymphocytes, including the steps of (1) measuring an expression level of GATA3 in a sample of lymphocytes collected from a subject, (2) incubating the sample of lymphocytes with a Th2 cytokine inhibitor if the expression level of GATA3 in the sample of lymphocytes is above a control value, and (3) incubating the sample of lymphocytes with a polynucleotide vector to transform the sample of lymphocytes to produce transformed lymphocytes.

In such an embodiment, the Th2 cytokine inhibitor is selected from the group consisting of an anti-IL4 antibody, an anti-IL5 antibody, and an anti-IL13 antibody, or combinations thereof. More specifically, the anti-IL4 antibody is dupilumab (Dupixent), the anti-IL5 antibody is mepolizumab, relizumab, or benralizumab, and an anti-IL13 antibody is trakolinumab.

In such an embodiment, the polynucleotide vector is a retroviral or lentiviral vector and encodes an anti-CD19 chimeric antigen receptor (CAR).

In such an embodiment, an activation step is included if a retrovirus is used to transduce the polynucleotide vector into the lymphocytes. The activation step is accomplished by stimulating the sample of lymphocytes with at least one T cell stimulating agent such as an anti-CD3 antibody, an anti-CD28 antibody, or Interleukin-2, or combinations thereof.

The transformed or transduced lymphocytes can be cultured for a maximum of 72 hours, 48 hours, or 36 hours if there is a need to generate a final immunotherapy product from the transformed or transduced lymphocytes in an expedited manner.

### Example 3

This example details the processes employed to develop a 24-color flow cytometry panel for confirming the scRNAseq and scATACseq GATA3+CD8+ results of Example 1.

First, markers ideal for identifying T cell subsets were determined. Importantly, to best evaluate the effects of the tumor microenvironment (TME) on promoting CAR-T cell differentiation along a certain lineage, it was determined that a combination of transcription factors and cytokines would need to be assayed. Given the amount of involvement of intra-cellular (IC), intra-nuclear (IN) staining and incompatibility of evaluating cytokine and transcription factors in one assay, identification of the ideal markers and the optimization needed to develop a flow cytometry protocol for consistently using such a combination of markers for evaluating the effects of the tumor microenvironment (TME) on promoting CAR-T cell differentiation was challenging.

Table 2 below outlines the markers used to identify T cell subsets.

TABLE 2
Markers used to identify T cell subsets

| Subset | Marker(s) |
|---|---|
| CAR | a-FCM63 (and aTGFbRII in case of armored CAR) |
| Th1 | CD194−/CD196−/CD183+/IFNg+/IL-2+ |
| Th2 | CD194+/CD196−/CD183−/GATA3+/IL-4+ |
| Th17 | CD194+/CD196+/CD183−/RORgt+/IL-17+ |
| T-regs | CD127low/—/CD25+/FoxP3+/IL-10+ |
| Naïve-Memory | Naïve: (CD62L+CD45RA+CCR7+) |
| | Central memory (CD62L+CD45RA−CCR7+), |
| | Effector memory (CD62L−CD45RA− CCR7−) |
| | Terminal effector memory (CD62L−CD45RA+CCR7−) |

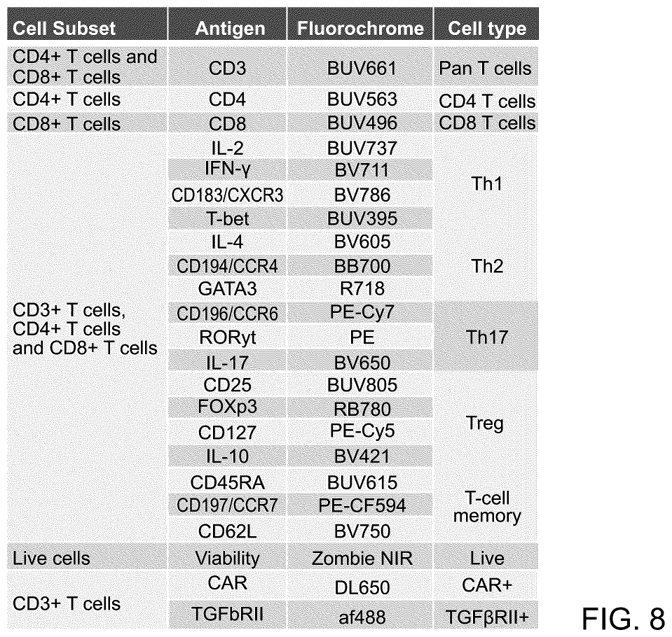

The final panel iteration with fluorophore assignment and marker specificity is shown in FIG. 8.

Next, polarized T cells were used to optimize the 24-color panel and analyze samples where the T cell immunophenotype was already known. The protocol for generation of polarized T cells used as positive controls in panel optimization is a follows: (1) Th1 cells were activated with anti-CD3 (1.23 ug/ml), anti-CD28 (1.66 ug/ml), anti-IL-10 (5 ug/ml), anti-IL-4 (5 ug/ml), IL-2 (305 IU/ml), and IL-12 (2.5 ng/ml); (2) Th2 cells were activated with anti-CD3 (1.23 ug/ml), anti-CD28 (1.66 ug/ml), anti-IL-10 (5 ug/ml), anti-IFNg (5 ug/ml), IL-2 (305 IU/ml), and IL-4 (12.5 ng/ml); (3) Th17 cells were activated with anti-CD3 (1.23 ug/ml), IL-23 (50 ng/ml), IL-1b (50 ng/ml), and were not activated with anti-CD28, and no IL-2 was added until day 6; and (4) Treg cells were activated with anti-CD3 (1.23 ug/ml), anti-CD28 (1.66 ug/ml), IL-2 (305 IU/ml), and TGF-β (5 ng/ml). These polarized cells served as controls for various purposes, including optimal clone selection, fluorophore selections, biological controls, antibody-reagent performance, gating, quality control of antibody cocktail(s), and batch control.

Next, since the 24-color panel includes a variety of cell surface and intracellular markers, optimization was performed to identify the best cell surface clones and fluorophores compatible with a transcription fixation/permeabilization buffer, as well as the best clones and fluorophores for detecting intracellular cytokine and intranuclear transcription factor antigens compatible with the transcription fixation/permeabilization buffer.

More specifically, two different fixation/permeabilization buffers were employed: (1) a cytokine fixation/permeabilization buffer, and (2) a transcription factor fixation/permeabilization buffer. Cytokine fixation/permeabilization buffers use milder permeabilization agents to access cytoplasmic antigens, while transcription factor fixation/permeabilization buffers use stronger agents to penetrate the nuclear membrane and reach transcription factors.

Titrations for the antibodies against the intracellular cytokines in the 24-color panel were initially optimized using cytokine fixation/permeabilization buffer to find an ideal titration/concentration for each of the antibodies against the intracellular cytokines in the 24-color panel. However, it was discovered that such a titration/concentration, though well-suited for use of the antibody with a cytokine fixation/permeabilization buffer, was not ideal for use of the antibody with a transcription factor fixation/permeabilization buffer. As such, the titration process for each of the antibodies against the intracellular cytokines in the 24-color panel was fine-tuned to identify the ideal titration/concentration for use of these antibodies in the transcription factor fixation/permeabilization buffer, using the initial titration/concentration for each antibody as obtained from cytokine permeabilization buffer as a starting point. As the titrations were lowered, ideal titrations for use in the transcription factor fixation/permeabilization buffer were identified for each of the antibodies against the intracellular cytokines.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments/aspects have been illustrated and described, it will be appreciated that various changes can be made without departing from the spirit and scope of the disclosure.

1. A method for manufacturing transformed lymphocytes, comprising:
measuring an expression level of GATA3 in a sample of lymphocytes collected from a subject;
reducing an activity of or an amount of GATA3+ CD8+ cells from the sample of lymphocytes if the expression level of GATA3 in the sample of lymphocytes is above a control value; and
incubating the sample of lymphocytes with a polynucleotide vector to transform the sample of lymphocytes to produce transformed lymphocytes.

2. The method of claim 1, wherein reducing the activity or the amount of GATA3+ CD8+ cells from the sample of lymphocytes comprises depleting cells expressing CD45RO from the sample of lymphocytes.

3. The method of claim 2, wherein depleting the cells expressing CD45RO from the sample of lymphocytes comprises:
contacting the sample of lymphocytes with an anti-hCD45RO antibody and bead conjugate so as to generate a bead-bound population of cells expressing CD45RO; and
separating the bead-bound population of cells expressing CD45RO from the sample of lymphocytes.

4. Method of claim 1, wherein reducing the activity or the amount of GATA3+ CD8+ cells from the sample of lymphocytes comprises incubating the sample of lymphocytes with a Th2 cytokine inhibitor, and wherein the Th2 cytokine inhibitor is selected from the group consisting of an anti-IL4 antibody, an anti-IL5 antibody, and an anti-IL13 antibody.

5. (canceled)

6. (canceled)

7. The method of claim 1, further comprising activating the sample of lymphocytes before incubating the sample of lymphocytes with the polynucleotide vector by stimulating the sample of lymphocytes with at least one T cell stimulating agent, wherein the at least one T cell stimulating agent comprises an anti-CD3 antibody, an anti-CD28 antibody, or Interleukin-2, and combinations thereof, and wherein the activating is carried out in a closed system.

8. (canceled)

9. (canceled)

10. (canceled)

11. (canceled)

12. (canceled)

13. The method of claim 1, further comprising, administering the transformed lymphocytes to a subject in need thereof or freezing the transformed lymphocytes.

14. The method of claim 1, wherein the polynucleotide vector is a viral vector, and wherein the viral vector is a retroviral vector or a lentiviral vector.

15. (canceled)

16. The method of claim 1, wherein the polynucleotide vector encodes a chimeric antigen receptor (CAR) or a T cell receptor (TCR), and wherein the CAR or the TCR recognizes a tumor antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, Fc receptor-like protein 5, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, TACI, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), EphA2, GPC2, CD133, LGR5, TROP2, CD146, CLDN3, CLDN4, CLDN6, CLDN9, CLDN18.2, CD70, TnMUC1, Alkaline phosphatase (placental type), MUC16, MUC17, MART1, Pmel17, Melanoma-associated chondroitin sulfate proteoglycan, as well as any derivate or variant of these antigens.

17. (canceled)

18. (canceled)

19. A population of cells prepared by the method of claim 1.

20. A pharmaceutical composition comprising the population of cells of claim 19.

21. A method for administering T cells to a subject, comprising injecting to the subject a harvested sample prepared by the method of claim 1.

22. (canceled)

23. (canceled)

24. (canceled)

25. (canceled)

26. A method of predicting a likelihood of a relapse to a cancer in a patient treated with a cell therapy product comprising:
measuring a pre-treatment level of GATA3+CD8+ cells in a sample of lymphocytes collected from the subject pre-treatment;
determining the likelihood of relapse in the patient from the pre-treatment level of GATA3+CD8+ cells,
wherein an elevated pre-treatment level of GATA3+CD8+ cells as compared to a pre-treatment control value from patients with the same cancer and same prior lines of therapy, if any, is indicative of an increased likelihood of relapse in the patient, and
wherein the patient is treated with the cell therapy product after the measuring the pre-treatment level of GATA3+CD8+ cells.

27. The method of claim 26, wherein the cancer is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.

28. The method of claim 26, wherein the cell therapy product is CAR T or TCR T cell therapy that recognizes a target antigen, wherein the cell therapy product is autologous or allogeneic, and wherein the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), as well as any derivate or variant of these antigens.

29. (canceled)

30. (canceled)

31. A method of predicting a likelihood of a relapse to a cancer in a patient treated with a cell therapy product comprising:
measuring a pre-treatment Th1-Th2 index in a sample of lymphocytes collected from the subject pre-treatment;
determining the likelihood of relapse in the patient from the pre-treatment Th1-Th2 index,
wherein a pre-treatment Th1-Th2 index below 0.0 is indicative of an increased likelihood of relapse in the patient, and
wherein the patient is treated with the cell therapy product after the measuring the pre-treatment Th1-Th2 index.

32. The method of claim 31, wherein the cancer is (relapsed or refractory) diffuse large B-cell lymphoma (DLBCL) not otherwise specified, primary mediastinal large B-cell lymphoma, high grade B-cell lymphoma, DLBCL arising from follicular lymphoma, or mantle cell lymphoma.

33. The method of claim 31, wherein the cell therapy product is CAR T or TCR T cell therapy that recognizes a target antigen, wherein the cell therapy product is autologous or allogeneic, and wherein the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), as well as any derivate or variant of these antigens.

34. (canceled)

35. (canceled)

36. A method for treating a malignancy in a patient comprising:
measuring an expression level of GATA3 in a cell therapy product;
determining whether the patient should be administered an effective dose of the cell therapy product, or an effective dose of the cell therapy product and an inhibitor of a Th2 cytokine based on the measuring the expression level of GATA3; and
administering the effective dose of the cell therapy product, or the effective dose of the cell therapy product and the inhibitor of a Th2 cytokine based on the determining step,
wherein the patient is administered the effective dose of the cell therapy product if the expression level of GATA3 is at or below a control value, and wherein the patient is administered the effective dose of the cell therapy product and the inhibitor of a Th2 cytokine if the expression level of GATA3 is above the control value.

37. The method of claim 36, wherein the inhibitor of a Th2 cytokine is selected from the list consisting of an IL-4 antagonist, an IL-5 antagonist, and an IL-13 antagonist.

38. (canceled)

39. The method of claim 36, wherein the cell therapy product is CAR T or TCR T cell therapy that recognizes a target antigen, and wherein the target antigen is a tumor antigen, preferably, selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), CD123, CD133, CD138, CD19, CD20, CD22, CD23, CD24, CD25, CD30, CD33, CD34, CD4, CD40, CD44, CD56, CD79a, CD79b, CD123, FLT3, BCMA, SLAMF7, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFl)-l, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, MN-CA IX, MUC-1, mut hsp70-2, mutated p53, mutated ras, neutrophil elastase, NKG2D, Nkp30, NY-ESO-1, p53, PAP, prostase, prostate specific antigen (PSA), prostate-carcinoma tumor antigen-1 (PCTA-1), prostate-specific antigen protein, STEAP1, STEAP2, PSMA, RAGE-1, ROR1, RU1, RU2 (AS), surface adhesion molecule, survivin and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gpl20), GPC3 (Glypican 3), as well as any derivate or variant of these antigens.

40. (canceled)

41. (canceled)

42. (canceled)

43. (canceled)

44. (canceled)
